(19) United States
(12) Patent Application Publication
(10) Pub. No.: US 2022/0143269 A1
(43) Pub. Date: May 12, 2022

(54) METHOD OF CULTURING ANIMAL CELLS AND ENGINEERING TISSUE AND TISSUE-LIKE STRUCTURES

(71) Applicants: UNIVERSITÉ DE TECHNOLOGIE DE COMPIÈGNE, Compiègne (FR); CENTRE NATIONAL DE LA RECHERCHE SCIENTIFIQUE, Paris (FR)

(72) Inventors: Christophe EGLES, Compiègne (FR); Pascale VIGNERON, Saint-Martin-Longueau (FR); Claude-Olivier SARDE, Compiègne (FR)

(73) Assignees: UNIVERSITÉ DE TECHNOLOGIE DE COMPIÈGNE, Compiègne (FR); CENTRE NATIONAL DE LA RECHERCHE SCIENTIFIQUE, Paris (FR)

(21) Appl. No.: 17/439,132

(22) PCT Filed: Mar. 27, 2020
(86) PCT No.: PCT/EP2020/058848

§ 371 Date: Sep. 14, 2021

(30) Foreign Application Priority Data
Mar. 27, 2019 (EP) 19305388.1

(51) Int. Cl. A61L 27/36; C12N 5/00; C12N 1/12; A61K 35/33; A61L 27/38

(52) U.S. Cl. CPC A61L 27/3637; C12N 5/0068; C12N 1/125; A61L 2430/34; A61L 27/3895; C12N 2533/90; A61K 35/33

(57) ABSTRACT

Volvox-derived beads, a method for the production thereof, and their uses as a scaffold for animal cell culture, in a method of culturing animal cells and in a method of engineering tissue and tissue-like structures. Also, compositions and pharmaceutical compositions including engineered tissue and tissue-like structures and to the cosmetic and therapeutic uses thereof.

### FIELD OF INVENTION

The present invention relates to the field of cell culture and tissue engineering. The present invention thus relates to a method for obtaining tissue-like structures comprising the use of inactivated Volvox colonies. The present invention also relates to compositions and pharmaceutical compositions comprising engineered tissue-like structures and to the cosmetic and therapeutic uses thereof.

### BACKGROUND OF INVENTION

Tissue engineering aims at producing tissue, or tissue-like structures, that may be used as suitable biological substitutes to restore, maintain or improve tissues or organs. Tissue engineering thus relies on the capacity to culture animal cells in conditions enabling the production of organized, cohesive tissue or tissue-like structures.

Tissue engineering generally entails cell co-cultures and/or addition to the animal cell culture of molecular elements of various origins in order to reconstitute organized tissue architectures. The latter approach has been pursued with the aim of mimicking native cellular environment, i.e., the biological, biophysical, and biomechanical conditions encountered in vivo, in order to induce the most efficient cell seeding and growth (Carletti et al., Methods Mol Biol. 2011; 695:17-39). Indeed, it has become more and more obvious that such conditions may greatly stimulate the intrinsic capacity of seeded animal cells to develop tissue-like structures (Quintana et al., Tissue Eng Part A. 2009 January; 15(1):45-54). However, the composition of the ideal support to be used for this purpose is still to be defined.

In the quest for the most adapted support, emphasis has been put on the adhesive properties of the support components, on which the cells can shore up and adhere. Trials have been conducted to identify adhesive compounds favoring animal cell implantation. Numerous natural biological materials, among which collagen, cellulose, chitosan, silk, alginate, hyaluronic acid, and peptide hydrogels, have thus been tested, either alone or in combination, with various fortunes. To date, the best results have been obtained with sugar matrix components supplemented with mammalian extracellular matrix (ECM) proteins or with pure type I collagen gels of animal origin. However, the use of animal products remains associated with ethical, legal, regulatory and safety concerns (O'Grady & Bordon, Adv Drug Deliv Rev. 2003 Nov. 28; 55(12):1699-1721). Some synthetic compounds or natural but non-animal compounds, including plant or algae biochemical compounds, have also been used with some success (Carletti et al., Methods Mol Biol. 2011; 695:17-39). However, most of the time, such compounds enter in artificial reconstitutions built from purified and often modified biochemicals, which are sometimes supplemented with animal material. The use of such artificial reconstitutions may thus be hindered by the above-listed concerns.

In parallel, the efficient elaboration of multidimensional architecture has been found to rely on the generation of individuals geometrical entities formed of cells entrapped or adhesive to a biocompatible material. Such entities may either be treated as independent individual structures and used as passive cell support, e.g., in reactor systems, or be considered as building bricks in the elaboration of a multilayered architecture. In this respect, alginate has long been used as the favorite material to generate cell trapping beads that may be used, for example, in bioreactors (Dufresne et al., in L. de Bartolo & A. Bader, 2012, CRC Press (USA); de Vos et al., Adv Drug Deliv Rev. 2014 April; 67-68:15-34) or even to generate animal implants (Simmons et al., Bone. 2004 August; 35(2):562-9). Under well-defined conditions, alginate mixed together with individual cells of different origin can generate reproducible spherical structures in which the cells may be efficiently mired. However, this trapping process is somehow damaging to the cells as the most deeply buried cells have a limited access to the nutrients provided externally.

Therefore, there remains a need for a support allowing the seeded animal cells to adhere and proliferate in an efficient manner, thus inducing the formation of individuals entities encompassing both the animal cells and the support, that may then be used for the engineering of tissue-like structures suitable for use in a subject.

The present invention relates to Volvox-derived beads, consisting of inactivated Volvox colonies, which the Applicants found to be a biocompatible support to which animal cells readily adhere, thus allowing the proliferation of said animal cells and the formation of individual three-dimensional entities, i.e., aggregates, comprising animal cells and at least one Volvox-derived bead. The present invention also relates to a method for the production of the Volvox-derived beads, and their use as a support for animal cell culture, in particular in a method for culturing animal cells and in a method for engineering tissue and tissue-like structures. Notably, the tissue-like structures engineered with the Volvox-derived beads of the invention are suitable for use in a subject, either for cosmetic or for therapeutic purposes.

### SUMMARY

One object of the invention is a method of culturing animal cells comprising culturing the animal cells in a culture medium comprising Volvox-derived beads, or a scaffold for animal cell culture comprising or consisting of Volvox-derived beads, said Volvox-derived beads consisting of inactivated Volvox colonies, wherein the animal cells adhere to the Volvox-derived beads and proliferate around said Volvox-derived beads, thereby forming animal cell aggregates comprising animal cells and at least one Volvox-derived bead.

In one embodiment, said method comprises:
first contacting the animal cells to be cultured with Volvox-derived beads, or a scaffold for animal cell culture comprising or consisting of Volvox-derived beads, and incubating them in culture medium;
then transferring the animal cells and Volvox-derived beads, or scaffold for animal cell culture comprising or consisting of Volvox-derived beads, to a culture vessel and culturing the animal cells in the presence of Volvox-derived beads or a scaffold for animal cell culture comprising or consisting of Volvox-derived bead.

In one embodiment, the animal cells to be cultured and the Volvox-derived beads are contacted in a ratio of about 1.10⁶ animal cells for about 0.2 cm³ to about 4 cm³ Volvox-derived beads.

In one embodiment, the animal cells to be cultured are human cells, preferably human primary cells.

Another object of the invention is a free animal cell aggregate comprising animal cells and at least one Volvox-derived bead, said Volvox-derived bead consisting of an inactivated Volvox colony.

Another object of the invention is a method of engineering tissue or a tissue-like structure, comprising:
culturing animal cells in a culture medium comprising Volvox-derived beads, or a scaffold for animal cell culture comprising or consisting of Volvox-derived beads, according to the method of the invention, thereby obtaining free animal cell aggregates according to the invention;
transferring and culturing said animal cell aggregates on culture inserts for tissue or tissue-like engineering and culturing said animal cell aggregates, thereby obtaining tissue or a tissue-like structure.

Another object of the invention is a composition comprising tissue or a tissue-like structure engineered with Volvox-derived beads, said Volvox-derived beads consisting of inactivated Volvox colonies.

Another object of the invention is a cosmetic use of the composition of the invention as a soft tissue filler.

Another object of the invention is a pharmaceutical composition comprising tissue or a tissue-like structure engineered with Volvox-derived beads, said Volvox-derived beads consisting of inactivated Volvox colonies, and at least one pharmaceutically acceptable excipient.

Another object of the invention is the pharmaceutical composition of the invention for use in the treatment of tissue loss or injury. In one embodiment, said tissue loss or injury is a post-surgical and/or post-traumatic tissue loss or injury.

Another object of the invention is a method of producing Volvox-derived beads, comprising:
culturing Volvox algae in order to obtain Volvox colonies;
isolating the Volvox colonies; and
inactivating the Volvox colonies, thereby producing Volvox-derived beads consisting of inactivated Volvox colonies; and
optionally dehydrating the Volvox-derived beads, thereby obtaining dehydrated Volvox-derived beads consisting of inactivated and dehydrated Volvox colonies.

In one embodiment, the Volvox colonies are inactivated in ethanol at a concentration expressed in volume/volume percent (v/v) ranging from about 60% to about 80%, preferably from about 65% to about 75%, for at least about 1 h, preferably at least about 2 h, at about 4° C.

In one embodiment, the method of producing Volvox-derived beads of the invention further comprises dehydrating the Volvox-derived beads, thereby obtaining dehydrated Volvox-derived beads consisting of inactivated and dehydrated Volvox colonies.

Another object of the invention is a scaffold for animal cell culture comprising dehydrated Volvox-derived beads, said Volvox-derived beads consisting of inactivated and dehydrated Volvox colonies.

The present invention also relates to a composition or solid support comprising Volvox-derived beads, said Volvox-derived beads consisting of inactivated Volvox colonies.

### Definitions

In the present invention, the following terms have the following meanings:
“About” preceding a figure encompasses plus or minus 10%, or less, of the value of said figure. It is to be understood that the value to which the term “about” refers is itself also specifically, and preferably, disclosed.
“Animal cells” refer to cells originating from a metazoan. Accordingly, cells of the Volvox alga are not animal cells. In one embodiment, the animal cells are cells originating from a vertebrate, which may be a fish, an amphibian, a reptile, a bird, or a mammal. In a particular embodiment, the animal cells are mammalian cells, preferably human cells.
“Allogeneic” or “allogenic” refers to any material (e.g., cells, cell aggregates, tissue or tissue-like structures) obtained or derived from a different subject of the same species than the subject to whom/which the material is to be introduced. Two or more subjects are said to be allogeneic to one another when the genes at one or more loci are not identical. In some aspects, allogeneic material from subjects of the same species may be sufficiently unlike genetically to interact antigenically.
“Autologous” refers to any material (e.g., cells, cell aggregates, tissue or tissue-like structures) obtained or derived from the same subject to whom/which it is later to be re-introduced.
“Cosmetic use” in the present invention refers to the use of tissue or tissue-like structure, composition or pharmaceutical composition of the invention for esthetic purposes. Examples of esthetic purposes include, without being limited to, lip augmentation or wrinkle smoothing. In the meaning of the present invention, cosmetic use excludes any use for therapeutic purposes, such as the prevention or treatment of a defect, disease or disorder, in particular the prevention or treatment of tissue loss or injury.
“Free” when referring to an animal cell or an animal cell aggregate means that said animal cell or said animal cell aggregate is isolated, i.e., is not part of an organism. In one embodiment, a “free” or “isolated” animal cell is an animal cell which has been separated from its natural environment. In this embodiment, the terms “free” or “isolated” include gross physical separation from the natural environment, such as, for example, removal from the donor animal, and alteration of the cell's relationship with its neighboring cells or with cells it is in direct contact by dissociation. In one embodiment, a “free” or “isolated” animal cell aggregate is an animal cell aggregate obtained in vitro. In one embodiment, a “free” or “isolated” animal cell aggregate is an animal cell aggregate obtained in vitro that is floating in the culture medium in which it is formed, i.e., that does not adhere to the culture vessel in which it is formed.
“Pharmaceutically acceptable excipient” refers to an excipient that does not produce an adverse, allergic or other untoward reaction when administered to a subject, preferably a human. Pharmaceutically acceptable excipients include any and all solvents, dispersion media, coatings, antibacterial and antifungal agents, isotonic and absorption delaying agents and the like. For human administration, pharmaceutical compositions comprising at least one pharmaceutically acceptable excipient should meet sterility, pyrogenicity, general safety and purity standards as required by regulatory Offices, such as, for example, the US Food and Drug Administration (FDA) or the European Medicines Agency (EMA).
“Pharmaceutical composition” refers to a composition comprising at least one pharmaceutically acceptable excipient, and as such, suitable for administration to a subject, in particular to a human subject. In the meaning of the present invention, a pharmaceutical composition may have a cosmetic use and may also be used for the prevention and/or treatment of a defect, disease, or disorder, in particular the prevention or treatment of tissue loss or injury.
“Primary cells” refer to cells taken directly from a living, non-cancerous tissue, such as, for example, cells obtained from a tissue sample of a subject or from a biopsy of a subject.
“Scaffold” refers to a structure or carrier matrix to which cells can attach and/or on which cells can proliferate. In the present invention, a scaffold for animal cell culture can also be referred to as a support for animal cell culture or as a substrate for animal cell culture. In one embodiment, the scaffold (or support or substrate) of the invention is biocompatible. In other words, in one embodiment, the scaffold (or support or substrate) of the invention is suitable for use in a method for obtaining or engineering biomaterial, e.g., animal cell aggregates or tissue or tissue-like structure, that may be used in a subject. In one embodiment, the scaffold (or support or substrate) of the invention is dehydrated or desiccated. In other words, in one embodiment, the scaffold (or support or substrate) of the invention presents as a powder.
“Soft tissue” refers to the tissue of an animal body that is not hardened or calcified, i.e., that is not bone, teeth, nails, hair, and cartilage. One of the functions of soft tissue within an animal body is to connect, support, or surround bone and internal organs. In one embodiment, soft tissue includes, without being limited to, tendons, ligaments, fascia, skin, mucosa, fibrous tissues, fat, synovial membranes, muscles, nerves and blood vessels.
“Subject” refers to an animal, preferably to a mammal, more preferably to a human. In one embodiment, the subject is a male. In another embodiment, the subject is a female. In one embodiment, the subject is an adult. In another embodiment, the subject is a child. In one embodiment, the subject is a patient, i.e., an animal, preferably a mammal, more preferably a human, who/which is awaiting the receipt of, or is receiving, medical care or was/is/will be the subject of a medical procedure that may induce tissue loss or injury, or is monitored for the development or progression of a disease or condition or of a treatment that may induce tissue loss or injury. In one embodiment, the subject is a human patient who is treated and/or monitored for the development or progression of a disease or condition that may induce tissue loss or injury, such as, for example, scleroderma, muscle diseases, vascular diseases, fibrosis or cirrhosis. In one embodiment, the subject has a preexisting disease or condition that may induce tissue loss, such as, for example ulcers, scleroderma, fibrosis or cirrhosis. In one embodiment, the subject is undergoing a treatment that may induce tissue loss or injury, such as, for example, cancer radiation treatment. In one embodiment, the subject has undergone surgery. In one embodiment, the subject has suffered trauma such as a wound or a burn.
“Tissue-like structure(s)” refer to three-dimensional structure(s) resulting from the culture of animal cells and mimicking the organization of animal tissue.
“Tissue loss or injury” as used herein refers to the loss or injury of tissue, such as soft tissue, cartilage or bone. Tissue loss or injury may be inborn (e.g., the consequence of a failure of the tissue to develop normally) or may be the consequence of a disease (e.g., scleroderma), a treatment (e.g., cancer radiation treatment), a surgery or a trauma (e.g., a wound or a burn).
“Treating” or “treatment” refers to therapeutic treatment, to prophylactic or preventative treatment or measures, or to both therapeutic treatment and prophylactic or preventative treatment or measures, wherein the object is to prevent, slow down (lessen) or repair tissue loss or injury. In one embodiment, the object is to lessen or repair post-surgical or post-traumatic defects. A subject is successfully “treated” if, after receiving tissue or tissue-like structures engineered with the Volvox-derived beads according to the invention, the subject shows at least one of the following: observable and/or measurable repair of the tissue loss or injury; stable filling of the surrounding tissue; relief to some extent of one or more of the symptoms associated with the tissue loss or injury; reduced morbidity and mortality; and/or improvement in quality of life issues. The above parameters for assessing successful treatment and improvement in the disease are readily measurable by routine procedures familiar to the physician.
“Untreated culture vessel”, e.g., untreated culture dish, refers to a culture vessel, e.g., a culture dish, that did not undergo the chemical treatment or the coating commonly applied to culture vessels in order to enhance cell attachment to the vessels. In the field of cell culture, “treated culture vessel” commonly refers to a culture vessel, such as a plate or a dish, usually made of polystyrene, that underwent a chemical treatment or a coating in order to enhance the attachment of adherent cells to the vessel. Indeed, polystyrene is a very hydrophobic polymer to which cells have difficulty attaching. Chemical treatment or coating with biological materials such as collagen or heparin sulfate, or with basic synthetic polymers such as poly-D-lysine, is thus applied to render the polystyrene surface more hydrophilic, resulting in the so-called “treated culture vessel”.

### DETAILED DESCRIPTION

The present invention relates to Volvox-derived beads comprising or consisting of inactivated alga colonies of the genus Volvox or inactivated alga colonies related to the Volvox genus. In the present invention, said Volvox-derived beads may also be referred to as Volvox-derived carrier beads (VCBs).

According to one embodiment, an alga colony is considered related to the Volvox genus if it is phylogenetically related to the Volvox genus.

According to one embodiment, an alga colony related to the Volvox genus comprises cells having a nucleotide sequence comprising the sequence of the 18S ribosomal RNA (rRNA), internal transcribed spacer region 1 (ITS1), 5.8S ribosomal RNA (rRNA), internal transcribed spacer region 2 (ITS2) and/or 28S ribosomal RNA (rRNA) at least about 50%, 55%, 60%, 65%, 70%, or 75%, preferably at least about 80%, 85%, 90%, 91%, 92%, 93%, 94%, 95%, 95.5%, 96%, 96.5%, 97%, 97.5%, 98%, 98.5%, 99% or 99.5% identical to a nucleotide sequence comprising the sequence of the 18S rRNA, ITS1, 5.8S rRNA, ITS2 and/or 28S rRNA of a cell of the genus Volvox.

In one embodiment, an alga colony related to the Volvox genus comprises cells having a nucleotide sequence of the internal transcribed spacer region 2 (ITS2) at least about 50%, 55%, 60%, 65%, 70%, or 75%, preferably at least about 80%, 85%, 90%, 91%, 92%, 93%, 94%, 95%, 95.5%, 96%, 96.5%, 97%, 97.5%, 98%, 98.5%, 99% or 99.5% A identical to a nucleotide sequence of ITS2 of a cell of the genus Volvox.

In one embodiment, an inactivated alga colony of the genus Volvox or an inactivated alga colony related to the Volvox genus may also be referred to as an inactivated Volvox colony.

According to the present invention, one Volvox-derived bead comprises or consists of one inactivated Volvox colony.

The present invention also relates to dehydrated Volvox-derived beads, which can also be referred to as desiccated, dried or powdered Volvox-derived beads.

According to the present invention, one dehydrated Volvox-derived bead comprises or consists of one inactivated and dehydrated Volvox colony.

Appearing to have evolved from the unicellular Chlamydomonas, algae of the Volvox genus are fresh water green algae which form round multicellular colonies. A Volvox colony, or coenobium, is a hollow ball composed of 500-50 000 cells, mostly somatic cells each with a pair of flagella. The somatic cells are organized in a single layer at the surface of the colony, with the flagella facing outward allowing the Volvox colony to swim. The inner part of the colony consists of a core mucosal structure, i.e., an extracellular matrix, essentially composed of polysaccharides and hydroxyproline-rich glycoproteins (HRGPs). More than 95% of the volume of a Volvox colony is thus formed by its extracellular matrix (ECM).

The prototype of the genus, Volvox carteri, is a spherical flagellated colony which architecture can be subdivided in four distinct zones: the flagellar zone (FZ); the boundary zone (BZ); the cellular zone (CZ); and the deep zone (DZ) (Kirk et al., J Cell Sci. 1986 February; 80:207-31). The deep zone (DZ), which corresponds to the most internal part of the alga, is a bead of amorphous polysaccharide-rich components, essentially supplemented with the hydroxyproline-rich glycoprotein (HRGPs) pherophorin-S. (Godl et al., EMBO J. 1997 Jan. 2; 16(1):25-34). The deep zone only contains six to eight reproductive cells named gonidia and is covered on its external surface by a cellular zone (CZ) made of a monolayer of about 2000 to 4000 bi-flagellated somatic cells, which adhere to a proteinaceous scaffold. The scaffold is devoid of the complex polysaccharide network generally found in plants (Domozych & Domozych, Front Plant Sci. 2014 Nov. 18; 5:649) and contains two major HRGPs, SSG 185 and pherophorin II. The SSG 185 protein is a fibrous structural element that creates regular honeycomb-like compartments within the ECM of the colony through covalent crosslink of its polysaccharide chains (Hoist et al., Eur J Biochem. 1989 May 1; 181(2):345-50). The presence of numerous cysteine residues in SSG 185 may also contribute to its intermolecular linkages (Ertl et al., J Cell Biol. 1989 December; 109(6 Pt 2):3493-501). Pherophorin II also contains a hydroxyproline-rich rod-like domain surrounded by globular regions (Sumper et al., EMBO J. 1993 March; 12(3):831-6). Its organization is similar to the Solanaceae lectin class of extensins (Lamport et al., Plant Physiol. 2011 May; 156(1):11-19). Extensins present a carbohydrate-binding ability in plant ECM, a role analogous to that of collagen in animals. (Kieliszewski and Shpak, Cell Mol Life Sci. 2001 September; 58(10):1386-98).

According to the invention, a Volvox colony is a spherical structure which comprises Volvox cells, mostly Volvox somatic cells, and mucus. In a Volvox colony, the mucus constitutes the inner part of the spherical structure and is surrounded by the somatic cells organized in a single layer at the surface of the spherical structure. In the meaning of the present invention, mucus is defined as an amorphous extracellular matrix, essentially composed of polysaccharides and hydroxyproline-rich glycoproteins (HRGPs).

In one embodiment, an alga colony of the genus Volvox comprises from about 500 cells to about 60 000 cells, preferably from about 1000 cells to 20 000 cells, more preferably from about 2000 cells to about 4000 cells.

In one embodiment, the diameter of an alga colony of the genus Volvox ranges from about 50 μm to about 750 μm, preferably from about 150 μm to about 500 μm.

In one embodiment, the Volvox-derived beads of the invention comprise or consist of inactivated alga colonies of the genus Volvox selected from the group comprising or consisting of Volvox carteri, Volvox africanus, Volvox amboensis, Volvox aureus, Volvox barberi, Volvox capensis, Volvox chaos, Volvox dissipatrix, Volvox fertilis, Volvox gigas, Volvox globator, Volvox merrillii, Volvox migulae, Volvox obversus, Volvox ovalis, Volvox perglobator, Volvox pilula, Volvox pocockiae, Volvox polychlamys, Volvox poonaensis, Volvox powersii, Volvox prolificus, Volvox rousseletii, Volvox spermatosphaera, Volvox tertius, and mixes thereof.

In one embodiment, the Volvox-derived beads of the invention comprise or consist of inactivated alga colonies of the genus Volvox selected from the group comprising or consisting of Volvox carteri, Volvox globator, and mixes thereof.

In one embodiment, the Volvox-derived beads of the invention comprise or consist of inactivated alga colonies of Volvox carteri.

As mentioned hereinabove, Volvox-derived beads (also referred to as Volvox-derived carrier beads) may comprise or consist of inactivated alga colonies related to the genus Volvox.

According to one embodiment, an alga colony is related to the genus Volvox if it is phylogenetically related to the genus Volvox.

In one embodiment, phylogenetic relatedness is determined through analysis and comparison of a nucleotide sequence of the alga colony to a nucleotide sequence of a species of the Volvox genus.

In one embodiment, the nucleotide sequences to be analyzed and compared are the nucleotide sequences of the 18S ribosomal RNA (rRNA), the internal transcribed spacer region 1 (ITS1), the 5.8S ribosomal RNA (rRNA), the internal transcribed spacer region 2 (ITS2) and/or the 28S ribosomal RNA (rRNA). In one embodiment, the nucleotide sequences to be analyzed and compared are the nucleotide sequences of the ITS1 and/or the ITS2. In one embodiment, the nucleotide sequences to be analyzed and compared are the nucleotide sequences of the ITS2.

In one embodiment, the nucleotide sequences are analyzed and compared using pairwise alignments and/or multiple alignments.

Methods to determine phylogenetic relatedness from pairwise alignments of nucleotide sequences are well-known to the person skilled in the art and include, without being limited to, neighbor-joining, maximum parsimony, distance matrix methods, maximum likelihood, and Bayesian inference. Computer programs that can be used to determine phylogenetic relatedness using such methods are well-known to the person skilled in the art and include for example PAUP* 4.0 versions (Sinauer Associates, Sunderland, Mass.).

In one embodiment, phylogenetic relatedness is determined through the analysis and comparison of the nucleotide sequence of the ITS1 and/or the ITS2 of the alga colony to the nucleotide sequence of the ITS1 and/or the ITS2 of a species of the Volvox genus.

In one embodiment, phylogenetic relatedness is determined as described in Coleman, 1999 (Proc Natl Acad Sci USA. 1999 Nov. 23; 96(24): 13892-13897).

In one embodiment, an alga colony is related to the genus Volvox if the distance between said alga colony and a species of the Volvox genus lies within the maximum level of pairwise distance between the two most distant species of the genus Volvox.

According to one embodiment, relatedness of an alga colony to the genus Volvox is assessed through the comparison of the nucleotide sequence of its 18S ribosomal RNA (rRNA), internal transcribed spacer region 1 (ITS1), 5.8S ribosomal RNA (rRNA), internal transcribed spacer region 2 (ITS2) and/or 28S ribosomal RNA (rRNA) to the nucleotide sequence of the 18S ribosomal RNA (rRNA), internal transcribed spacer region 1 (ITS1), 5.8S ribosomal RNA (rRNA), internal transcribed spacer region 2 (ITS2) and/or 28S ribosomal RNA (rRNA) of a species of the Volvox genus.

In embodiment, the nucleotide sequence of the 18S rRNA, ITS1, 5.8S rRNA, ITS2 and/or 28S rRNA of a species of the genus Volvox is the nucleotide sequence of the 18S rRNA, ITS1, 5.8S rRNA, ITS2 and/or 28S rRNA of Volvox carteri or Volvox globator, preferably of Volvox carteri.

In one embodiment, an alga colony is related to the genus Volvox if the nucleotide sequence of its 18S rRNA, ITS1, 5.8S rRNA, ITS2 and/or 28S rRNA is at least about 50%, 55%, 60%, 65%, 70%, or 75%, preferably at least about 80%, 85%, 90%, 91%, 92%, 93%, 94%, 95%, 95.5%, 96%, 96.5%, 97%, 97.5%, 98%, 98.5%, 99% or 99.5% identical to the nucleotide sequence of the 18S rRNA, ITS1, 5.8S rRNA, ITS2 and/or 28S rRNA of a species of the genus Volvox as described hereinabove, preferably Volvox carteri or Volvox globator, more preferably Volvox carteri.

In one embodiment, the nucleotide sequence of a species of the Volvox genus comprising the sequence of the 18S rRNA, ITS1, 5.8S rRNA, ITS2 and/or 28S rRNA is a nucleotide sequence of the ITS2 of Volvox carteri selected from the group comprising or consisting of SEQ ID NO: 1 to 5.

In one embodiment, the nucleotide sequence of a species of the Volvox genus comprising the sequence of the 18S rRNA, ITS1, 5.8S rRNA, ITS2 and/or 28S rRNA is the nucleotide sequence of the ITS2 of Volvox globator SEQ ID NO: 6.

The identity between two nucleotide sequences is a direct function of the number of matches (i.e., identical positions). For example, if half of the positions in two sequences are identical, the two sequences are 50% identical; if 90% of the positions are identical, the two sequences are 90% identical. The homology between two nucleotide sequences refers to a common ancestor, as expressed by a high percentage of identity. Thus, the terms “identical” or “homologous”, when used in a relationship between the sequences of two or more nucleic acid molecules (or nucleotide sequences), refer to the degree of sequence relatedness between nucleic acid molecules (or nucleotide sequences), as determined by the number of matches between strings of two or more nucleotide residues. “Identity” measures the percent of identical matches between the smaller of two or more sequences with gap alignments (if any) addressed by a particular mathematical model or computer program (i.e., “algorithms”) Identity can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucleic Acids Res. 1984 Jan. 11; 12(1 Pt 1):387-95; Genetics Computer Group, University of Wisconsin, Madison, Wis.), BLASTN, and FASTA (Altschul et al., J Mol Biol. 1990 Oct. 5; 215(3):403-10). The well-known Smith Waterman algorithm may also be used to determine identity.

In the present invention, an inactivated Volvox colony is a Volvox colony wherein all the Volvox cells are dead. In other words, an inactivated Volvox colony according to the present invention is no longer living. According to the present invention, an inactivated Volvox colony retains its spherical structure.

In one embodiment, a Volvox colony is inactivated if said colony is no longer floating and/or rolling when incubated in a medium allowing the culture of Volvox algae (i.e., a suitable culture medium) in conditions allowing the culture of Volvox algae (i.e., suitable culture conditions).

Medium and conditions suitable for the culture of Volvox algae are well-known. For example, Volvox algae may be incubated in VT medium (as defined hereafter) at about 22° C. with a day/night cycle of about 12 h/12 h and lighting of about 13 000 lux and about 37 W/m².

In one embodiment, a Volvox colony is inactivated if incubation of said Volvox colony in fresh culture medium does not revivify the Volvox colony, thereby demonstrating that no living cells are present in the inactivated Volvox colony.

As mentioned hereinabove, an object of the invention is dehydrated Volvox-derived beads, also referred to as desiccated, dried or powdered Volvox-derived beads.

In one embodiment of the present invention, dehydrated Volvox-derived beads present as a dry powder, i.e., a Volvox-derived bead dry powder.

According to the present invention, dehydrated Volvox-derived beads, i.e., a Volvox-derived bead dry powder, may be rehydrated.

The Applicants observed that upon rehydration, the morphological and structural properties of the rehydrated Volvox-derived beads are similar to that of Volvox-derived beads prior to dehydration.

The invention also relates to a method of producing Volvox-derived beads, said method comprising:
culturing Volvox algae in order to obtain Volvox colonies;
optionally isolating the Volvox colonies; and
inactivating the Volvox colonies, thereby producing Volvox-derived beads.

In one embodiment, the method comprises:
culturing Volvox algae in order to obtain Volvox colonies;
optionally isolating the Volvox colonies;
optionally washing the isolated Volvox colonies;
inactivating the Volvox colonies, thereby producing Volvox-derived beads; and
optionally washing the isolated Volvox colonies.

According to the invention, Volvox algae are cultured in a culture medium and in conditions suitable for the formation of Volvox colonies.

Culture media and conditions suitable for the formation of Volvox colonies are well-known to the person skilled in the art.

Examples of culture media that may be used in the present invention include, without being limited to, Volvox medium and modified Volvox media from UTEX (Austin, Tex.) such as, for example, HEPES medium, MES-Volvox medium, P49 medium, Volvox-dextrose medium. Examples of appropriate media for the culture of Volvox algae also include VT medium comprising 500 μmol/L Ca(NO₃)₂; 235 μmol/L Na₂-β-glycerophosphate; 162 μmol/L MgSO₄; 670 μmol/L KCl; 0.07 nmol/L vitamin B12; 0.41 nmol/L biotin; 30 nmol/L thiamine; 3.8 mmol/L glycylglycine; 8 μmon Na₂EDTA.2H₂O; 2.2 nmol/L FeCl₃; 0.55 μmon MnCl₂; 0.11 μmol/L ZnSO₄; 0.05 μmol/L CoCl₂; 0.036 μmon Na₂MoO₄; with a pH buffered at 7.5.

In one embodiment, the Volvox algae, preferably Volvox carteri, are grown in VT medium.

Examples of culture conditions suitable for the formation of Volvox colonies include, without being limited to:
a temperature ranging from about to 18° C. to about 26° C., preferably from about 20° C. to about 24° C., more preferably about 22° C.; and
a day/night alternation of about 10 h/14 h, 11 h/13 h, 12 h/12 h, 13 h/11 h or 14 h/10 h, preferably about 12 h/12 h; and
a lighting ranging from about 10 000 lux to about 15 000 lux, preferably from about 11 000 lux to about 14 000 lux, more preferably about 13 000 lux and ranging from about 30 W/m² to about 42 W/m², preferably from about 33 W/m² to about 40 W/m², more preferably about 37 W/m².

In one embodiment, the Volvox algae, preferably Volvox carteri, are grown at a temperature of about 22° C., with a day/night alternation of about 12 h/12 h and a lighting of about 13 000 lux and about 37 W/m².

In one embodiment, the Volvox algae, preferably Volvox carteri, are grown for at least about 2, 3, 4, 5, 6, 7, 8, 9, 10, 11, 12, 13, 14, 15 or more days.

In one embodiment, the Volvox algae, preferably Volvox carteri, are grown for at least about 1, 2, 3 or more weeks.

According to the invention, Volvox colonies are isolated from the culture medium after an appropriate number of colonies has been reached.

In one embodiment, Volvox colonies are isolated from the culture medium after at least about 1×10³, 5×10³, 1×10⁴, 5×10⁴, or 10⁵ colonies have been obtained.

In one embodiment, Volvox colonies are isolated from the culture medium after at least about 0.25, 0.50, 0.75 or 1 cm³ of colonies have been obtained.

According to the invention, methods for isolating Volvox colonies are methods allowing the isolation of Volvox colonies without damaging the structure of the colonies.

Examples of said methods include, without being limited to, filtration, centrifugation, decanting, sieving and the like.

According to one embodiment, Volvox colonies are sieved on a stainless-steel screen with a porosity of about 50 μM, 100 μM, 150 μM, or 200 μM.

In one embodiment, Volvox colonies, preferably Volvox carteri colonies, are sieved on a stainless-steel screen with a porosity of about 100 μM.

Following isolation, Volvox colonies may optionally be washed in buffer, for example with PBS or any other suitable buffer.

Thus, in one embodiment, the method of the invention further comprises washing the Volvox colonies obtained after isolation from the culture medium.

In one embodiment, isolated Volvox colonies, preferably Volvox carteri colonies, are washed in PBS and centrifuged at 300 g for about 5 min.

According to the invention, Volvox colonies are inactivated so that all the Volvox cells of the colonies are killed while the colonies retain their spherical structure. For example, Volvox colonies may be inactivated through their incubation in ethanol, formaldehyde, methanol, acetone, glutaraldehyde and the like.

In one embodiment, Volvox colonies are incubated in ethanol, in formaldehyde, in methanol, in acetone, or in glutaraldehyde.

In one embodiment, Volvox colonies are incubated in ethanol at a concentration expressed in volume/volume percent (v/v) ranging from about 50% to about 100%, preferably from about 60% to about 80%, more preferably from about 65% to about 75%.

In one embodiment, Volvox colonies are incubated in ethanol at a concentration expressed in volume/volume percent (v/v) of about 60%, 65%, 70%, 75%, 80%, 85% or 90%.

In one embodiment, Volvox colonies are incubated in ethanol at a concentration expressed in volume/volume percent (v/v) of about 70%.

In one embodiment, the incubation of Volvox colonies in ethanol as described hereinabove lasts for at least about 30 min, preferably at least about 1 h and more preferably at least about 2 h, preferably at about 4° C.

In one embodiment, the method of producing Volvox-derived beads of the invention comprises:
culturing Volvox algae, preferably Volvox carteri, in order to obtain Volvox colonies, wherein the Volvox algae, preferably Volvox carteri, are grown in culture medium, preferably VT medium as defined hereinabove, at a temperature of about 22° C., with a day/night alternation of about 12 h/12 h and a lighting of about 13 000 lux and about 37 W/m²;
isolating the Volvox colonies, wherein the Volvox colonies, preferably Volvox carteri colonies, are sieved on a stainless-steel screen with a porosity of about 100 μM;
optionally washing the isolated colonies in buffer, preferably PBS; and
inactivating the Volvox colonies, wherein the Volvox colonies, preferably Volvox carteri colonies, are incubated in ethanol at a concentration expressed in volume/volume percent (v/v) ranging from about 60% to about 80%, preferably from about 65% to about 75%, for at least about 1 h, preferably at least about 2 h, preferably at about 4° C.;
   thereby producing Volvox-derived beads.

Following inactivation, Volvox colonies may optionally be washed in buffer, for example with PBS or any other suitable buffer.

Thus, in one embodiment, the method of the invention further comprises washing the inactivated Volvox colonies obtained after incubation in ethanol, in formaldehyde, in methanol, in acetone, or in glutaraldehyde as described hereinabove.

In one embodiment, the method of producing Volvox-derived beads of the invention comprises:
culturing Volvox algae, preferably Volvox carteri, in order to obtain Volvox colonies, wherein the Volvox algae, preferably Volvox carteri, are grown in culture medium, preferably VT medium as defined hereinabove, at a temperature of about 22° C., with a day/night alternation of about 12 h/12 h and a lighting of about 13 000 lux and about 37 W/m²;
isolating the Volvox colonies, wherein the Volvox colonies, preferably Volvox carteri colonies, are sieved on a stainless-steel screen with a porosity of about 100 μM;
optionally washing the isolated colonies in buffer, preferably PBS;
inactivating the Volvox colonies, wherein the Volvox colonies, preferably Volvox carteri colonies, are incubated in ethanol at a concentration expressed in volume/volume percent (v/v) ranging from about 60% to about 80%, preferably from about 65% to about 75%, for at least about 1 h, preferably at least about 2 h, preferably at about 4° C.; and
optionally washing the Volvox-derived beads in buffer, preferably PBS;
   thereby producing Volvox-derived beads.

According to one embodiment, the Volvox-derived beads of the invention are obtained, or susceptible to be obtained, by the method as described hereinabove.

According to one embodiment of the invention, the Volvox-derived beads of the invention are dehydrated or desiccated. In one embodiment of the invention, the dehydrated or desiccated Volvox-derived beads thus present as a dry powder. Dehydration methods are well-known to the skilled person.

For example, Volvox-derived beads may be dehydrated through incubation in ethanol at a concentration of about 100% (v/v) or incubation in ethanol at a concentration raising from about 50% to about 100% (v/v) followed by ethanol evaporation. Volvox-derived beads may also be dehydrated through incubation in a desiccator or through lyophilization, also known as freeze-drying, for example after incubation in ethanol.

According to one embodiment, Volvox-derived beads are dehydrated by incubation in ethanol at a concentration expressed in volume/volume percent (v/v) ranging from about 50% to about 100%. In one embodiment, Volvox-derived beads are dehydrated by incubation in ethanol at a concentration expressed in volume/volume percent (v/v) raising progressively from about 50% to about 100%. In one embodiment, Volvox-derived beads are dehydrated by incubation in ethanol at a concentration expressed in volume/volume percent (v/v) of about 100%.

In one embodiment, the Volvox-derived beads are incubated in ethanol, particularly in ethanol at a concentration (v/v) of about 100%, until they are at least 90% dehydrated. In one embodiment, the Volvox-derived beads are incubated in ethanol, particularly in ethanol at a concentration (v/v) of about 100%, until complete dehydration. For example, the Volvox-derived beads are incubated in ethanol at a concentration (v/v) of about 100%, for at least about 10 min, 15 min, 20 min, 30 min, 45 min, 1 h, 2 h, 3 h, 4 h, 5 h, 6 h, 12 h, 18 h, 24 h, 30 h, 36 h, 42 h or 48 h.

In one embodiment, incubation in ethanol at a concentration (v/v) ranging from about 50% to about 100% is followed by ethanol evaporation. According to this embodiment, the ethanol is evaporated until at least 90% desiccation is reached, preferably until complete desiccation. Evaporation methods are well-known to the skilled person and include, without being limited to, vacuum drying, heating, liquid nitrogen freezing followed by ice sublimation (also known as freeze-drying or lyophilization).

Thus, in one embodiment, Volvox-derived beads are dehydrated by incubation in ethanol at a concentration (v/v) ranging from about 50% to about 100%, followed by lyophilization, also known as freeze-drying.

In one embodiment, following incubation in ethanol at a concentration (v/v) ranging from about 50% to about 100%, the Volvox-derived beads are washed, preferably in PBS or any other suitable buffer. In one embodiment, any residual buffer and/or ethanol is evaporated as described hereinabove, for example with vacuum drying, heating, or lyophilization.

According to one embodiment, Volvox-derived beads are dehydrated by lyophilization, also known as freeze-drying.

Another object of the invention is thus a method of producing dehydrated Volvox-derived beads comprising:
producing Volvox-derived beads as described hereinabove;
incubating the Volvox-derived beads in ethanol at a concentration (v/v) ranging from about 50% to about 100%, preferably of about 100%, as described hereinabove;
optionally washing the Volvox-derived beads in buffer, preferably PBS;
evaporating the residual ethanol and/or buffer from the Volvox-derived beads, preferably lyophilizing the Volvox-derived beads as described hereinabove;
   thereby producing dehydrated Volvox-derived beads.

In one embodiment, the method of producing dehydrated Volvox-derived beads comprises:
culturing Volvox algae, preferably Volvox carteri, in order to obtain Volvox colonies, wherein the Volvox algae, preferably Volvox carteri, are grown in culture medium, preferably VT medium as defined hereinabove, at a temperature of about 22° C., with a day/night alternation of about 12 h/12 h and a lighting of about 13 000 lux and about 37 W/m²;
isolating the Volvox colonies, wherein the Volvox colonies, preferably Volvox carteri colonies, are sieved on a stainless-steel screen with a porosity of about 100 μM;
optionally washing the isolated colonies in buffer, preferably PBS; and
inactivating the Volvox colonies, wherein the Volvox colonies, preferably Volvox carteri colonies, are incubated in ethanol at a concentration expressed in volume/volume percent (v/v) ranging from about 60% to about 80%, preferably from about 65% to about 75%, for at least about 1 h, preferably at least about 2 h, preferably at about 4° C., thereby producing Volvox-derived beads;
optionally washing the Volvox-derived beads in buffer, preferably PBS;
evaporating the residual ethanol and/or buffer, preferably lyophilizing the Volvox-derived beads;
   thereby producing dehydrated Volvox-derived beads.

In one embodiment, the method of producing dehydrated Volvox-derived beads comprises:
culturing Volvox algae, preferably Volvox carteri, in order to obtain Volvox colonies, wherein the Volvox algae, preferably Volvox carteri, are grown in culture medium, preferably VT medium as defined hereinabove, at a temperature of about 22° C., with a day/night alternation of about 12 h/12 h and a lighting of about 13 000 lux and about 37 W/m²;
isolating the Volvox colonies, wherein the Volvox colonies, preferably Volvox carteri colonies, are sieved on a stainless-steel screen with a porosity of about 100 μM;
optionally washing the isolated colonies in buffer, preferably PBS; and
inactivating the Volvox colonies, wherein the Volvox colonies, preferably Volvox carteri colonies, are incubated in ethanol at a concentration expressed in volume/volume percent (v/v) ranging from about 60% to about 80%, preferably from about 65% to about 75%, for at least about 1 h, preferably at least about 2 h, preferably at about 4° C., thereby producing Volvox-derived beads;
incubating the Volvox-derived beads in ethanol at a concentration (v/v) ranging from about 50% to about 100%, preferably of about 100%, for at least about 10 min;
optionally washing the Volvox-derived beads in buffer, preferably PBS;
evaporating the residual ethanol and/or buffer, preferably lyophilizing the Volvox-derived beads;
   thereby producing dehydrated Volvox-derived beads.

The present invention also relates to the use of the Volvox-derived beads or dehydrated Volvox-derived beads (i.e., Volvox-derived beads presenting as dry powder) as described hereinabove as a scaffold or support for animal cell culture.

Thus, another object of the invention is a scaffold or support for animal cell culture comprising or consisting of Volvox-derived beads or dehydrated Volvox-derived beads as described hereinabove.

In one embodiment, the scaffold or support for animal cell culture comprising Volvox-derived beads or dehydrated Volvox-derived beads as described hereinabove further comprises silk fibroin and/or extracellular matrix (ECM) proteins such as, for example, collagen, laminin or fibronectin.

In one embodiment, the scaffold or support for animal cell culture comprising Volvox-derived beads or dehydrated Volvox-derived beads as described hereinabove further comprises silk fibroin, collagen and/or other extracellular matrix (ECM) proteins such as laminin or fibronectin.

In one embodiment, the scaffold or support of the invention is dehydrated or desiccated. In other words, in one embodiment, the scaffold or support of the invention presents as a powder.

The present invention also relates to Volvox-derived beads or dehydrated Volvox-derived beads (i.e., Volvox-derived beads presenting as dry powder) as described hereinabove for use as a medicament, and in particular for use in wound healing.

Examples of wounds include, without being limited to, cuts, burns, abrasions, scrapes, grazes, lacerations, incisions.

Thus, another object of the invention is a composition comprising Volvox-derived beads (or Volvox-derived carrier beads), said Volvox-derived beads comprising or consisting of inactivated Volvox colonies, as described hereinabove.

In one embodiment, the composition comprises dehydrated Volvox-derived beads as described hereinabove.

In one embodiment, the composition is a solution. In one embodiment, the composition is an emulsion, such as an oil-in-water emulsion or a water-in-oil emulsion. In one embodiment, the composition is a balm, a cream, a gel, a lotion, a paste, an ointment, a salve, an unction, or an unguent.

In one embodiment, the composition is a pharmaceutical composition, further comprising at least one pharmaceutically acceptable excipient.

Pharmaceutically acceptable excipients that may be used in the pharmaceutical composition of the invention include non-toxic solid, semi-solid or liquid filler, diluent, encapsulating material or formulation auxiliary of any type.

In one embodiment, the composition or pharmaceutical composition comprises Volvox-derived beads or dehydrated Volvox-derived beads as described hereinabove in a proportion ranging from about 1% to about 10% by weight (weight/weight).

In one embodiment, the composition or pharmaceutical composition comprising Volvox-derived beads or dehydrated Volvox-derived beads as described hereinabove comprises at least one additional component, preferably selected from the group comprising or consisting of sucralfate, also named hexadeca-μ-hydroxytetracosahydroxy [μ8-[1,3,4,6-tetra-O-sulfo-β-Dfructofuranosyl-α-D-glucopyranoside tetrakis (hydrogen sulfato)8-)]]hexadecaaluminum, a copper salt, and a zinc salt.

Another object of the invention is a solid support comprising Volvox-derived beads (or Volvox-derived carrier beads), said Volvox-derived beads comprising or consisting of inactivated Volvox colonies, as described hereinabove.

In one embodiment, the solid support comprises dehydrated Volvox-derived beads as described hereinabove.

In one embodiment, the solid support comprises Volvox-derived beads or dehydrated Volvox-derived beads as described hereinabove in a proportion ranging from about 1% to about 10% by weight (weight/weight).

In one embodiment, the solid support is coated with a composition comprising Volvox-derived beads or dehydrated Volvox-derived beads as described hereinabove.

Examples of solid supports include, without being limited to, resins, dressings or wound dressings, patches, compresses, gauzes, medical plasters or wound plasters, bandages, band-aids, strips or adhesive strips, and casts.

Thus, in one embodiment, the solid support comprising Volvox-derived beads or dehydrated Volvox-derived beads is a resin, a dressing or a wound dressing, a patch, a compress, a gauze, a medical plaster or wound plaster, a bandage, a band-aid, a strip or adhesive strip, or a cast.

The present invention also relates to a method of culturing animal cells, said method comprising culturing the animal cells in a culture medium comprising Volvox-derived beads, or a scaffold for animal cell culture comprising or consisting of Volvox-derived beads, as described hereinabove.

According to one embodiment, the method of culturing animal cells of the invention comprises:
adding Volvox-derived beads, or a scaffold for animal cell culture comprising or consisting of Volvox-derived beads, as described hereinabove and the animal cells to be cultured in a culture medium; and
culturing the animal cells in the culture medium comprising Volvox-derived beads, or a scaffold for animal cell culture comprising or consisting of Volvox-derived beads.

As mentioned above, according to the present invention, one Volvox-derived bead comprises or consists of one inactivated Volvox colony.

In one embodiment, the Volvox-derived beads and the animal cells to be cultured are added to culture medium in a ratio of about 0.1 cm³ to about 5 cm³ of Volvox-derived beads for about 1.10⁶ animal cells, preferably in a ratio of about 0.2 cm³ to about 4 cm³ of Volvox-derived beads for about 1.10⁶ animal cells.

In one embodiment, the Volvox-derived beads and the animal cells to be cultured are added to culture medium in a ratio of about 1 cm³ of Volvox-derived beads for about 1.10⁶ animal cells.

In one embodiment, the Volvox-derived beads and the animal cells to be cultured are added to culture medium in a ratio of about 4.5×10² to about 15×10⁶ Volvox-derived beads for about 1.10⁶ animal cells, preferably in a ratio of about 1×10³ to about 1×10⁶ Volvox-derived beads for about 1.10⁶ animal cells.

In one embodiment, the Volvox-derived beads and the animal cells to be cultured are added to culture medium in a ratio of about 2×10⁴ to about 4×10⁵ Volvox-derived beads for about 1.10⁶ animal cells, preferably in a ratio of about 1×10⁵ Volvox-derived beads for about 1.10⁶ animal cells.

According to one embodiment, the method of culturing animal cells of the invention comprises:
adding dehydrated Volvox-derived beads, or a scaffold for animal cell culture comprising or consisting of dehydrated Volvox-derived beads, as described hereinabove and the animal cells to be cultured in a culture medium, thus rehydrating said dehydrated Volvox-derived beads; and
culturing the animal cells in the culture medium comprising rehydrated Volvox-derived beads, or a scaffold for animal cell culture comprising or consisting of rehydrated Volvox-derived beads.

In one embodiment, the method of culturing animal cells of the invention comprises:
adding the animal cells to be cultured in a culture medium;
adding the dehydrated Volvox-derived beads, or a scaffold for animal cell culture comprising or consisting of dehydrated Volvox-derived beads, as described hereinabove to the culture medium comprising the animal cells, thus rehydrating said dehydrated Volvox-derived beads; and
culturing the animal cells in the culture medium comprising rehydrated Volvox-derived beads, or a scaffold for animal cell culture comprising or consisting of rehydrated Volvox-derived beads.

In one embodiment, the method of culturing animal cells of the invention comprises:
adding the dehydrated Volvox-derived beads, or a scaffold for animal cell culture comprising or consisting of dehydrated Volvox-derived beads, as described hereinabove to a culture medium, thus rehydrating said dehydrated Volvox-derived beads;
adding the animal cells to be cultured to the culture medium comprising rehydrated Volvox-derived beads, or a scaffold for animal cell culture comprising or consisting of rehydrated Volvox-derived beads; and
culturing the animal cells in the culture medium comprising rehydrated Volvox-derived beads, or a scaffold for animal cell culture comprising or consisting of rehydrated Volvox-derived beads.

In one embodiment, the dehydrated Volvox-derived beads and the animal cells to be cultured are added to culture medium in a ratio of dehydrated Volvox-derived beads equivalent to from about 0.1 cm³ to about 5 cm³ of Volvox-derived beads for about 1.10⁶ animal cells, preferably in a ratio of dehydrated Volvox-derived beads equivalent to from about 0.2 cm³ to about 4 cm³ of Volvox-derived beads for about 1.10⁶ animal cells.

In one embodiment, the dehydrated Volvox-derived beads and the animal cells to be cultured are added to culture medium in a ratio of dehydrated Volvox-derived beads equivalent to about 1 cm³ of Volvox-derived beads for about 1.10⁶ animal cells.

In one embodiment, the dehydrated Volvox-derived beads and the animal cells to be cultured are added to culture medium in a ratio of dehydrated Volvox-derived beads equivalent to from about 4.5×10² to about 15×10⁶ Volvox-derived beads for about 1.10⁶ animal cells, preferably in a ratio of dehydrated Volvox-derived beads equivalent to from about 1×10³ to about 1×10⁶ Volvox-derived beads for about 1.10⁶ animal cells.

In one embodiment, the dehydrated Volvox-derived beads and the animal cells to be cultured are added to culture medium in a ratio of dehydrated Volvox-derived beads equivalent to from about 2×10⁴ to about 4×10⁵ Volvox-derived beads for about 1.10⁶ animal cells, preferably in a ratio of dehydrated Volvox-derived beads equivalent to about 1×10⁵ Volvox-derived beads for about 1.10⁶ animal cells.

In one embodiment, the dehydrated Volvox-derived beads and the animal cells to be cultured are added to culture medium in a ratio of about 5 mg to about 50 mg of dehydrated Volvox-derived beads for about 1.10⁶ animal cells, preferably in a ratio of about 20 mg of dehydrated Volvox-derived beads for about 1.10⁶ animal cells.

Virtually any type of animal cells may be cultured according to the method of the invention.

In one embodiment, the animal cells as described hereinabove are mammal cells. In another embodiment, the animal cells as described hereinabove are primate cells. In another embodiment, the animal cells as described hereinabove are human cells.

In one embodiment, the animal cells to be cultured with the method of the invention are primary animal cells. In another embodiment, the animal cells are immortalized, transformed or transfected animal cells.

In one embodiment, the animal cells to be cultured with the method of the invention are somatic or differentiated animal cells. In another embodiment, the animal cells to be cultured with the method of the invention are stem cells.

In one embodiment, the animal cells as described hereinabove are not human embryonic stem cells.

Examples of animal cells include, without being limited to, epithelial cells, neural cells, immune cells, bone cells, and soft tissue cells.

Examples of particular types of animal cells include, without being limited to, fibroblasts such as, for example, dermal fibroblasts or gingival fibroblasts, endothelial cells, smooth muscle cells, pancreas cells and hepatocytes.

In one embodiment, the animal cells to be cultured with the method of the invention are selected from the group comprising or consisting of fibroblasts, keratinocytes, adipocytes, epithelial cells, endothelial cells, smooth muscle cells, neural cells, pancreas cells, hepatocytes, and any mixes thereof.

In one embodiment, the animal cells to be cultured with the method of the invention are selected from the group comprising or consisting of fibroblasts, keratinocytes, adipocytes, epithelial cells, endothelial cells, smooth muscle cells, neural cells and any mixes thereof.

In one embodiment, the animal cells to be cultured with the method of the invention are selected from the group comprising or consisting of fibroblasts, keratinocytes, adipocytes, and any mixes thereof.

In one embodiment, the animal cells to be cultured with the method of the invention are fibroblasts, preferably human fibroblasts, even more preferably primary human fibroblasts.

In one embodiment, the animal cells to be cultured with the method of the invention are obtained from a sample of tissue previously taken from a subject, in particular from a biopsy previously taken from a subject. Examples of such tissue samples, or biopsies, include, without being limited to, sample or biopsies of skin, of bone, of nerve, of muscle, of pancreas, and of liver.

According to the present invention, any culture medium suitable for the culture of animal cells as described hereinabove may be used in the method of the invention.

Culture media that may be used in the method of the invention include natural media and synthetic media, such as, for example, serum-containing media, serum-free media, xeno-free media notably for human cell culture, protein-free media, chemically defined media.

According to the present invention, the culture medium may be supplemented with additional substances such as salts, carbon sources, amino acids, serum and serum components, vitamins, minerals, reducing agents, buffering agents, lipids, nucleosides, antibiotics, attachment factors, and growth factors.

Examples of suitable culture media include, without being limited to Basal Medium Eagle (BME), Eagle's Minimum Essential Medium (EMEM), Minimum Essential Medium (MEM), Dulbecco's Modified Eagles Medium (DMEM), Ham's F-10, Ham's F-12, Kaighn's modified Ham's F-12, DMEM/F-12, McCoy's 5A medium and complete culture medium.

As used herein, the term “complete culture medium” refers to a basal culture medium as listed hereinabove supplemented with additional substances which may include, without being limited to, fetal calf serum (FCS) also referred to as fetal bovine serum (FBS), amino acids and optionally specific growth factors and hormones such as, for example, EGF (epidermal growth factor), KGF (keratinocyte growth factor), insulin, HGF (hepatocyte growth factor), thyroid extracts and/or VEGF (vascular endothelial growth factor).

Suitable culture media according to the present invention also include media suitable for the culture of a particular type of animal cells, such as, for example, culture media suitable for the culture of primary cells (e.g., Medium 106, Gibco or Medium 199), culture media suitable for the culture of stem cells (e.g., Essential 8 Medium, Gibco), culture media suitable for the culture of neuronal cells (e.g., Neurobasal Medium, Gibco), culture media suitable for the culture of keratinocytes (e.g., Epilife, Gibco), culture media suitable for the culture of fibroblasts, for example gingival fibroblasts (e.g., Fibrolife, Lifeline technology), culture media suitable for the culture of endothelial cells (e.g., Endothelial Cell Media, Promocell).

In one embodiment, the culture medium used in the method of culturing animal cells of the invention is not conducive to the survival and growth of Volvox algae. In other words, in one embodiment, the culture medium used in the method of culturing animal cells of the invention would not allow the survival and growth of living Volvox algae if they were added to said medium.

Selecting a medium suitable to the culture of animal cells is well-known to those skilled in the art. Thus, the suitable culture medium to be used for the culture of animal cells with the method of the invention will be apparent to those skilled in the art and will depend on the animal cells to be cultured.

In one embodiment, the culture medium used in the method of the invention is selected from the group comprising or consisting of Eagle's Minimum Essential Medium (EMEM), Minimum Essential Medium (MEM), Dulbecco's Modified Eagles Medium (DMEM), Epilife medium, Fibrolife medium, and M199 (medium 199).

According to one embodiment, the animal cells to be cultured are first contacted with Volvox-derived beads, or a scaffold for animal cell culture comprising or consisting of Volvox-derived beads, and incubated in a first volume of culture medium as described hereinabove. Without wishing to be bound to any theory, the Applicants suggest that such a step may help ensure that the animal cells adhere to the Volvox-derived beads, or to the scaffold for animal cell culture comprising or consisting of Volvox-derived beads.

Thus, in one embodiment, the method of the invention comprises:
contacting the animal cells to be cultured with Volvox-derived beads, or a scaffold for animal cell culture comprising or consisting of Volvox-derived beads, and incubating them in a first volume of culture medium;
transferring the animal cells and Volvox-derived beads, or scaffold for animal cell culture comprising or consisting of Volvox-derived beads, to a culture vessel comprising a second volume of culture medium; and
culturing the animal cells in the presence of Volvox-derived beads or a scaffold for animal cell culture comprising or consisting of Volvox-derived beads.

As mentioned hereinabove, selecting a medium suitable to the culture of animal cells is well-known to the skilled persons in the art. Thus, the culture medium to be used for the culture of animal cells with the method of the invention will be apparent to those skilled in the art.

In one embodiment, the first volume of culture medium is smaller than the second volume of culture medium.

According to one embodiment of the invention, the first volume of culture medium corresponds to a volume of less than about 1000 mL, 900 mL, 800 mL, 700 mL, 600 mL, 500 mL, 400 mL, 300 mL, 200 mL or 100 mL.

In one embodiment, the first volume of culture medium corresponds to a volume of less than about 200 mL, 150 mL, 100 mL, 50 mL, 20 mL, 10 mL, 5 mL or 2 mL.

In one embodiment, the first volume of culture medium corresponds to a volume of less than about 30 mL, 25 mL, 20 mL, 15 mL, 10 mL, 5 mL, 2 mL or 1 mL.

In one embodiment, the first volume of culture medium corresponds to a volume of at most about 100 mL, 200 mL, 300 mL, 400 mL, 500 mL, 600 mL, 700 mL, 800 mL, 900 mL or 1000 mL.

In one embodiment, the first volume of culture medium corresponds to a volume of at most about 2 mL, 5 mL, 10 mL, 15 mL, 20 mL, 50 mL, 100 mL, 150 mL, or 200 mL.

In one embodiment, the first volume of culture medium corresponds to a volume of at most about 1 mL, 2 mL, 5 mL, 10 mL, 15 mL, 20 mL, 25 mL or 30 mL.

According to one embodiment of the invention, the first volume of culture medium corresponds to a volume ranging from about 0.5 mL to about 1000 mL, preferably from about 1 mL to about 500 mL, more preferably from about 5 mL to about 200 mL.

According to one embodiment of the invention, the animal cells to be cultured and the Volvox-derived beads, or a scaffold for animal cell culture comprising or consisting of Volvox-derived beads, are incubated in a first volume of culture medium as described hereinabove at a temperature suitable for the culture of the animal cells and for at least about 15 min, 30 min, 45 min, 60 min, 75 min, 90 min, 105 min or 120 min, preferably for at least about 30 min, more preferably for at least about 45 min. In another embodiment, the animal cells to be cultured and the Volvox-derived beads, or a scaffold for animal cell culture comprising or consisting of Volvox-derived beads, are incubated in a first volume of culture medium as described hereinabove in conditions suitable for the culture of the animal cells and for at most about 15 min, 30 min, 45 min, 60 min, 75 min, 90 min, 105 min or 120 min, preferably for at most about 90 min.

In another embodiment, the animal cells to be cultured and the Volvox-derived beads, or a scaffold for animal cell culture comprising or consisting of Volvox-derived beads, are incubated in a first volume of culture medium as described hereinabove at a temperature suitable for the culture of the animal cells and for about 15 min, 30 min, 45 min, 60 min, 75 min, 90 min, 105 min or 120 min, preferably for about 30 min, more preferably for about 45 min.

According to the present invention, conditions suitable for the first incubation of animal cells include parameters such as temperature and levels of CO₂. According to the present invention, said parameters, in particular temperature and levels of CO₂ are selected depending on the animal cells to be cultured with the method of the invention. Selecting culture conditions suitable to the culture of animal cells is well-known to those skilled in the art. Thus, the suitable culture conditions to be used for the culture of animal cells with the method of the invention will be apparent to those skilled in the art and will depend on the animal cells to be cultured.

For example, in one embodiment, the animal cells to be cultured, in particular human cells, and the Volvox-derived beads, or a scaffold for animal cell culture comprising or consisting of Volvox-derived beads, are incubated in a first volume of culture medium as described hereinabove at about 37° C. in about 5% CO₂.

In one embodiment, the animal cells to be cultured are contacted with the Volvox-derived beads in a ratio of about 1.10⁶ animal cells for about 0.1 cm³ to about 5 cm³ of Volvox-derived beads, preferably in a ratio of about 1.10⁶ animal cells for about 0.2 cm³ to about 4 cm³ of Volvox-derived beads.

In one embodiment, the animal cells to be cultured are contacted with the Volvox-derived beads in a ratio of about 1.10⁶ animal cells for about 1 cm³ of Volvox-derived beads.

In one embodiment, the animal cells to be cultured are contacted with the Volvox-derived beads in a ratio of about 1.10⁶ animal cells for about 4.5×10² to about 15×10⁶ Volvox-derived beads, preferably in a ratio of about 1.10⁶ animal cells for about 1×10³ to about 1×10⁶ Volvox-derived beads.

In one embodiment, the animal cells to be cultured are contacted with the Volvox-derived beads in a ratio of about 1.10⁶ animal cells for about 2×10⁴ to about 4×10⁵ Volvox-derived beads, preferably in a ratio of about 1.10⁶ animal cells for about 1×10⁵ Volvox-derived beads.

In one embodiment, the method of the invention comprises:
contacting the animal cells to be cultured with Volvox-derived beads, or a scaffold for animal cell culture comprising or consisting of Volvox-derived beads, wherein about 1.10⁶ animal cells and about 1 cm³ of Volvox-derived beads are added to a first volume of culture medium, preferably said first volume of culture medium corresponds to a volume of less than about 200 mL, 150 mL, 100 mL, 50 mL, 20 mL, 10 mL, 5 mL or 2 mL, and incubating them for at least about 45 min;
transferring the animal cells and Volvox-derived beads, or scaffold for animal cell culture comprising or consisting of Volvox-derived beads, to a vessel comprising a second volume of culture medium; and
culturing the animal cells in the presence of Volvox-derived beads or a scaffold for animal cell culture comprising or consisting of Volvox-derived beads.

In one embodiment, the animal cells to be cultured are first contacted with rehydrated Volvox-derived beads, or a scaffold for animal cell culture comprising or consisting of rehydrated Volvox-derived beads, wherein said rehydrated Volvox-derived beads are obtained upon addition of dehydrated Volvox-derived beads, or a scaffold for animal cell culture comprising or consisting of dehydrated Volvox-derived beads, to the culture medium.

In one embodiment, the dehydrated Volvox-derived beads, or a scaffold for animal cell culture comprising or consisting of dehydrated Volvox-derived beads, are added to the culture medium before the addition of animal cells to said culture medium.

In one embodiment, the dehydrated Volvox-derived beads, or a scaffold for animal cell culture comprising or consisting of dehydrated Volvox-derived beads, are added to the culture medium after the addition of animal cells to said culture medium. In one embodiment, the dehydrated Volvox-derived beads, or a scaffold for animal cell culture comprising or consisting of dehydrated Volvox-derived beads, and the animal cells are added to the culture medium concomitantly.

For example, in one embodiment, the method of the invention comprises:
adding dehydrated Volvox-derived beads, or a scaffold for animal cell culture comprising or consisting of dehydrated Volvox-derived beads, to a first volume of culture medium, thereby rehydrating said dehydrated Volvox-derived beads;
contacting the animal cells to be cultured with the rehydrated Volvox-derived beads, or a scaffold for animal cell culture comprising or consisting of rehydrated Volvox-derived beads, and incubating them in the first volume of culture medium;
transferring the animal cells and Volvox-derived beads, or scaffold for animal cell culture comprising or consisting of Volvox-derived beads, to a culture vessel comprising a second volume of culture medium; and
culturing the animal cells in the presence of Volvox-derived beads or a scaffold for animal cell culture comprising or consisting of Volvox-derived beads.

In one embodiment, the dehydrated Volvox-derived beads and the animal cells to be cultured are added to culture medium in a ratio of dehydrated Volvox-derived beads equivalent to from about 0.1 cm³ to about 5 cm³ of Volvox-derived beads for about 1.10⁶ animal cells, preferably in a ratio of dehydrated Volvox-derived beads equivalent to from about 0.2 cm³ to about 4 cm³ of Volvox-derived beads for about 1.10⁶ animal cells.

In one embodiment, the dehydrated Volvox-derived beads and the animal cells to be cultured are added to culture medium in a ratio of dehydrated Volvox-derived beads equivalent to about 1 cm³ of Volvox-derived beads for about 1.10⁶ animal cells.

In one embodiment, the dehydrated Volvox-derived beads and the animal cells to be cultured are added to culture medium in a ratio of dehydrated Volvox-derived beads equivalent to from about 4.5×10² to about 15×10⁶ Volvox-derived beads for about 1.10⁶ animal cells, preferably in a ratio of dehydrated Volvox-derived beads equivalent to from about 1×10³ to about 1×10⁶ Volvox-derived beads for about 1.10⁶ animal cells.

In one embodiment, the dehydrated Volvox-derived beads and the animal cells to be cultured are added to culture medium in a ratio of dehydrated Volvox-derived beads equivalent to from about 2×10⁴ to about 4×10⁵ Volvox-derived beads for about 1.10⁶ animal cells, preferably in a ratio of dehydrated Volvox-derived beads equivalent to about 1×10⁵ Volvox-derived beads for about 1.10⁶ animal cells.

In one embodiment, the dehydrated Volvox-derived beads and the animal cells to be cultured are added to culture medium in a ratio of about 5 mg to about 50 mg of dehydrated Volvox-derived beads for about 1.10⁶ animal cells, preferably in a ratio of about 20 mg of dehydrated Volvox-derived beads for about 1.10⁶ animal cells.

In one embodiment, the method of the invention comprises:
adding from about 5 mg to about 50 mg dehydrated Volvox-derived beads to a first volume of culture medium, preferably said first volume of culture medium corresponds to a volume of less than about 200 mL, 150 mL, 100 mL, 50 mL, 20 mL, 10 mL, 5 mL or 2 mL, thus rehydrating said dehydrated Volvox-derived beads;
contacting the animal cells to be cultured with the rehydrated Volvox-derived beads, wherein about 1.10⁶ animal cells are added to the first volume of culture medium comprising the rehydrated Volvox-derived beads, and incubating them for at least about 45 min;
transferring the animal cells and Volvox-derived beads, or scaffold for animal cell culture comprising or consisting of Volvox-derived beads, to a vessel comprising a second volume of culture medium; and
culturing the animal cells in the presence of Volvox-derived beads or a scaffold for animal cell culture comprising or consisting of Volvox-derived beads.

As mentioned hereinabove, virtually any type of animal cells may be cultured according to the method of the invention. It is commonly known in the art that suitable culture conditions, including parameters such as, for example, culture vessel, culture medium, volume of culture medium, frequency of medium renewal, temperature, levels of CO₂, and length of incubation, will depend on the animal cells to be cultured.

Examples of culture vessels include, without being limited to, plates, dishes, flasks, tubes and bioreactors.

In one embodiment, the animal cells and Volvox-derived beads, or scaffold for animal cell culture comprising or consisting of Volvox-derived beads, are added or transferred to a plate, a dish, a flask, a tube or a bioreactor.

In one embodiment, the animal cells and Volvox-derived beads, or scaffold for animal cell culture comprising or consisting of Volvox-derived beads, are added or transferred to an untreated culture vessel. In other words, in one embodiment, the animal cells and Volvox-derived beads, or scaffold for animal cell culture comprising or consisting of Volvox-derived beads, are added or transferred to a culture vessel that did not undergo a treatment to enhance cell attachment to the culture vessel.

According to one embodiment of the invention, the second volume of culture medium corresponds to a volume of less than about 5000 mL, 2000 mL, 1000 mL, 750 mL, 500 mL, 250 mL, 200 mL, 100 mL, 75 mL, 50 mL, 25 mL, 20 mL, 15 mL, 10 mL or 5 mL.

In on embodiment, the second volume of culture medium corresponds to a volume ranging from about 0.5 mL to about 5000 mL, preferably from about 1 mL to about 1000 mL, more preferably from about 2 mL to about 500 mL.

The suitable second volume of culture medium to be used for the culture of animal cells with the method of the invention will be apparent to those skilled in the art and will depend on the animal cells to be cultured and on the culture vessel to which the animal cells and Volvox-derived beads, or scaffold for animal cell culture comprising or consisting of Volvox-derived beads, are added or transferred.

According to one embodiment of the invention, after a first incubation in culture medium as described hereinabove, the animal cells to be cultured and the Volvox-derived beads, or a scaffold for animal cell culture comprising or consisting of Volvox-derived beads, are transferred to a culture vessel comprising the same culture medium.

In another embodiment, the animal cells to be cultured and the Volvox-derived beads, or a scaffold for animal cell culture comprising or consisting of Volvox-derived beads, are transferred to a culture vessel comprising a different culture medium.

Selecting culture conditions suitable for the animal cells to be cultured is well-known to those skilled in the art. Thus, the suitable culture conditions to be used for the culture of animal cells with the method of the invention, such as, for example, culture medium, volume of culture medium, frequency of medium renewal, temperature, levels of CO₂, and length of incubation, will be apparent to those skilled in the art and will depend on the animal cells to be cultured.

Examples of media suitable for the culture of the animal cells are mentioned hereinabove.

In one embodiment, the animal cells, in particular human cells, are cultured in presence of Volvox-derived beads, or a scaffold for animal cell culture comprising or consisting of Volvox-derived beads, for at least about 24 h, 36 h, 48 h, 60 h, 72 h or more.

In one embodiment, the animal cells, in particular human cells, are cultured in presence of Volvox-derived beads, or a scaffold for animal cell culture comprising or consisting of Volvox-derived beads, for at least about 1, 2, 3, 4, 5, 6, 7, 8, 9, 10, 11, 12, 13, 14, 15 or more day(s).

In one embodiment, the animal cells, in particular human cells, are cultured in presence of Volvox-derived beads, or a scaffold for animal cell culture comprising or consisting of Volvox-derived beads, for at least about 1, 2, 3, 4, 5, 6, 7, 8, or more week(s).

For example, in one embodiment, the animal cells, in particular human cells, are cultured in presence of Volvox-derived beads, or a scaffold for animal cell culture comprising or consisting of Volvox-derived beads, in a dish at about 37° C. in about 5% CO₂ for at least about 24 h and at most about 6 weeks.

In one embodiment, the method of the invention comprises:
contacting the animal cells to be cultured with Volvox-derived beads, or a scaffold for animal cell culture comprising or consisting of Volvox-derived beads, wherein about 1.10⁶ animal cells and about 1 cm³ of Volvox-derived beads are added to a first volume of culture medium, preferably said first volume of culture medium corresponds to a volume of less than about 200 mL, 150 mL, 100 mL, 50 mL, 20 mL, 10 mL, 5 mL or 2 mL, and incubating them for at least about 45 min;
transferring the animal cells and Volvox-derived beads, or scaffold for animal cell culture comprising or consisting of Volvox-derived beads, to an untreated culture vessel comprising a second volume of the same culture medium, preferably said second volume of culture medium ranges from about 1 mL to about 1000 mL; and
culturing the animal cells in the presence of Volvox-derived beads or a scaffold for animal cell culture comprising or consisting of Volvox-derived beads for at least about 24 h.

In one embodiment, the method of the invention comprises:
adding from of about 5 mg to about 50 mg dehydrated Volvox-derived beads to a first volume of culture medium, preferably said first volume of culture medium corresponds to a volume of less than about 200 mL, 150 mL, 100 mL, 50 mL, 20 mL, 10 mL, 5 mL or 2 mL, thus rehydrating said dehydrated Volvox-derived beads;
contacting the animal cells to be cultured with the rehydrated Volvox-derived beads, wherein about 1.10⁶ animal cells are added to the first volume of culture medium comprising the rehydrated Volvox-derived beads, and incubating them for at least about 45 min;
transferring the animal cells and Volvox-derived beads to an untreated culture vessel comprising a second volume of the same culture medium, preferably said second volume of culture medium ranges from about 1 mL to about 1000 mL; and
culturing the animal cells in the presence of Volvox-derived beads for at least about 24 h.

As mentioned hereinabove, the dehydrated Volvox-derived beads, or a scaffold for animal cell culture comprising or consisting of dehydrated Volvox-derived beads, may be added to the culture medium before, after or concomitantly with the addition of animal cells to said culture medium.

According to the method of the invention as described hereinabove, the animal cells to be cultured adhere to the Volvox-derived beads and proliferate around said Volvox-derived beads, thereby forming animal cell aggregates comprising or consisting of animal cells and at least one Volvox-derived bead. Within an aggregate, animal cells may either adhere directly to the Volvox-derived bead or indirectly, through attachment to other animal cells. Furthermore, aggregates comprising or consisting of animal cells and a Volvox-derived bead may congregate, thereby resulting in the formation of larger aggregates comprising or consisting of animal cells and Volvox-derived beads.

Indeed, without wishing to be bound by any theory, the Applicants have shown that animal cells adhere to Volvox-derived beads, and that said adhesion enables the growth and proliferation of the animal cells and results in the formation of animal cell aggregates comprising or consisting of animal cells surrounding and attaching to a Volvox-derived bead, either directly or indirectly through attachment to other animal cells within the aggregates.

Thus, in one embodiment, the present invention relates to a method of culturing animal cells as described hereinabove comprising culturing the animal cells in a culture medium comprising Volvox-derived beads, or a scaffold for animal cell culture comprising or consisting of Volvox-derived beads, as described hereinabove, wherein the animal cells adhere directly or indirectly to the Volvox-derived beads and proliferate around said Volvox-derived beads, thereby forming animal cell aggregates comprising or consisting of animal cells and at least one Volvox-derived bead, wherein the animal cells attach to the Volvox-derived beads or to other animal cells within the aggregates.

In one embodiment, the method of the invention as described hereinabove comprises:
contacting the animal cells to be cultured with Volvox-derived beads, or a scaffold for animal cell culture comprising or consisting of Volvox-derived beads, and incubating them in a first volume of culture medium;
transferring the animal cells and Volvox-derived beads, or scaffold for animal cell culture comprising or consisting of Volvox-derived beads, to a culture vessel comprising a second volume of culture medium; and
culturing the animal cells in the presence of Volvox-derived beads or a scaffold for animal cell culture comprising or consisting of Volvox-derived bead;
   thereby obtaining animal cell aggregates comprising or consisting of animal cells and at least one Volvox-derived bead.

In one embodiment, the method of the invention as described hereinabove comprises:
adding dehydrated Volvox-derived beads, or a scaffold for animal cell culture comprising or consisting of dehydrated Volvox-derived beads, to a first volume of culture medium, thereby rehydrating said dehydrated Volvox-derived beads;
contacting the animal cells to be cultured with the rehydrated Volvox-derived beads, or a scaffold for animal cell culture comprising or consisting of rehydrated Volvox-derived beads, and incubating them in the first volume of culture medium;
transferring the animal cells and Volvox-derived beads, or scaffold for animal cell culture comprising or consisting of Volvox-derived beads, to a culture vessel comprising a second volume of culture medium; and
culturing the animal cells in the presence of Volvox-derived beads or a scaffold for animal cell culture comprising or consisting of Volvox-derived bead;
   thereby obtaining animal cell aggregates comprising or consisting of animal cells and at least one Volvox-derived bead.

As mentioned hereinabove, the dehydrated Volvox-derived beads, or a scaffold for animal cell culture comprising or consisting of dehydrated Volvox-derived beads, may be added to the culture medium before, after or concomitantly with the addition of animal cells to said culture medium.

In one embodiment, the diameter of the animal cell aggregates of the invention is at least about 150 μm, 200 μm, 250 μm, 300 μm, 350 μm, 400 μm, 450 μm or 500 μm, preferably at least about 250 μm.

In one embodiment, the diameter of the animal cell aggregates of the invention ranges from about 150 μm to about 10 000 μm, preferably from about 200 μm to about 2000 μm, more preferably from about 250 μm to about 1000 μm.

In one embodiment, the animal cell aggregates of the invention comprise or consist of at least one, two, three, four, five or more layers of animal cells surrounding a Volvox-derived bead.

In one embodiment, the animal cell aggregates of the invention comprise or consist of animal cells and Volvox-derived beads in a ratio of at least about 2, 5, 10, 20, 30, 40, 50, 75, or 100 animal cells for one Volvox-derived bead.

In one embodiment, the animal cell aggregates of the invention comprise or consist of animal cells and Volvox-derived beads in a ratio of about 2 to about 500 animal cells for one Volvox-derived bead, preferably in a ratio of about 10 to about 200 animal cells for one Volvox-derived bead.

In one embodiment, the animal cell aggregates of the invention comprise or consist of more than one Volvox-derived beads, each surrounded by animal cells.

In one embodiment, the animal cell aggregates of the invention comprise or consist of at least 1, 2, 3, 4, or more Volvox-derived bead(s), each surrounded by a number of animal cells ranging from about 2 to about 500, preferably from about 10 to about 200.

The present invention also relates to an animal cell culture medium as described hereinabove comprising Volvox-derived beads, or a scaffold for animal cell culture comprising or consisting of Volvox-derived beads, as described hereinabove. In other words, another object of the invention is an animal cell culture medium comprising inactivated Volvox colonies according to the invention.

Examples of animal cell culture medium are provided hereinabove and include natural media and synthetic media, such as, for example, serum-containing media, serum-free media, xeno-free media notably for human cell culture, protein-free media, chemically defined media.

The present invention also relates to a free animal cell aggregate comprising or consisting of animal cells and at least one Volvox-derived bead.

According to one embodiment, said free animal cell aggregate refers to an animal cell aggregate comprising or consisting of animal cells and at least one Volvox-derived bead that is floating in the culture medium in which it is formed, i.e., animal cell aggregate comprising or consisting of animal cells and at least one Volvox-derived bead that does not adhere to the culture vessel in which it is formed.

Thus, the free animal cell aggregates of the invention may easily be collected from the culture medium and transferred to a new vessel for further use as described below.

In one embodiment, the free animal cell aggregates of the invention are obtained, or susceptible to be obtained, by the method of culturing animal cells as described hereinabove.

In one embodiment, the free animal cell aggregates of the invention have a diameter ranging from about 150 μm to about 10 000 μm, preferably from about 200 μm to about 2000 μm, more preferably from about 250 μm to about 1000 μm.

In one embodiment, the free animal cell aggregates of the invention comprise or consist of animal cells and Volvox-derived beads in a ratio of at least about 2, 5, 10, 20, 30, 40, 50, 75, or 100 animal cells for one Volvox-derived bead.

In one embodiment, the free animal cell aggregates of the invention comprise or consist of animal cells and Volvox-derived beads in a ratio of about 2 to about 500 animal cells for one Volvox-derived bead, preferably in a ratio of about 10 to about 200 animal cells for one Volvox-derived bead.

In one embodiment, the free animal cell aggregates of the invention comprise or consist of more than one Volvox-derived beads, each surrounded by animal cells.

Another object of the invention is a composition comprising a free animal cell aggregate comprising or consisting of animal cells and Volvox-derived beads as described hereinabove.

The present invention also relates to the use of the free animal cell aggregate or of the composition comprising a free animal cell aggregate as described hereinabove for engineering tissue-like structures, for engineering artificial organs. The present invention also relates to the free animal cell aggregate or the composition comprising a free animal cell aggregate as described hereinabove for a cosmetic use such as soft tissue filling, for use as a medicament and for use in the treatment of tissue loss or injury.

According to one embodiment, the free animal cell aggregate of the invention or a composition comprising said free animal cell aggregate(s) are obtained from cells of the subject on whom/which the free animal cell aggregate or the composition comprising said free animal cell aggregate(s) are to be used. Thus, in one embodiment, the free animal cell aggregate of the invention or a composition comprising said free animal cell aggregate(s) are obtained from autologous animal cells, preferably autologous human cells.

In another embodiment, the free animal cell aggregate of the invention or a composition comprising said free animal cell aggregate(s) are obtained from cells of a subject other than the subject on whom/which the free animal cell aggregate or the composition comprising said free animal cell aggregate(s) are to be used. Thus, in another embodiment, the free animal cell aggregate of the invention or a composition comprising said free animal cell aggregate(s) are obtained from allogenic animal cells, preferably allogenic human cells.

The present invention also relates to a method of engineering tissue, or a tissue-like structure, said method comprising transferring the free animal cell aggregate(s) of the invention on culture inserts for tissue engineering and culturing said animal cell aggregate(s), thereby obtaining tissue or a tissue-like structure.

In one embodiment, said free animal cell aggregate(s) of the invention are obtained, or susceptible to be obtained, by the method of culturing animal cells as described hereinabove.

In one embodiment, the method of engineering tissue, or a tissue-like structure, of the invention comprises:
culturing animal cells with the method of the invention as described hereinabove, thereby obtaining free animal cell aggregates comprising or consisting of animal cells and at least one Volvox-derived bead;
transferring said free animal cell aggregates of the invention on culture inserts for tissue engineering and culturing said animal cell aggregates, thereby obtaining tissue or a tissue-like structure.

According to the present invention, any culture inserts suitable for the engineering of tissue or tissue-like structures may be used in the method of the invention.

Example of suitable culture inserts include, without being limited to, polystyrene inserts fitted with polyester mesh bottoms, polyethylene terephthalate inserts, polycarbonate cell culture inserts, such as polycarbonate cell culture inserts with a pore size of 0.4 μm and a pore density of <0.85×10⁸ pores/cm², or with a pore size of 3 μm and a pore density of <1.70×10⁶ pores/cm².

In one embodiment, the cultures inserts used in the method of the invention are polycarbonate cell culture inserts.

In one embodiment, the free animal cell aggregates of the invention are cultured on culture inserts for tissue engineering in the culture medium in which the animal cells were cultured according to the method of culturing of the invention. In another embodiment, the free animal cell aggregates of the invention are cultured on culture inserts for tissue engineering in a culture medium different to that in which the animal cells were cultured according to the method of culturing of the invention.

Selecting culture conditions suitable for tissue engineering is well-known to those skilled in the art. Thus, the actual culture conditions to be used for the engineering of tissue or tissue-like structures with the method of the invention, such as, for example, culture medium, volume of culture medium in both the upper and lower compartments of the culture inserts, frequency of medium renewal, temperature, levels of CO₂, and length of incubation, will be apparent to those skilled in the art and will depend on the tissue or tissue-like structures to be engineered and on the culture inserts used in the method of the invention.

Example of media suitable for the engineering of tissue or tissue-like structures according to the method of the invention include the examples of culture media mentioned hereinabove.

In one embodiment, the animal cell aggregates are cultured on culture inserts for tissue engineering for at least about 24 h, 36 h, 48 h, 60 h, 72 h or more.

In one embodiment, the animal cell aggregates are cultured on culture inserts for tissue engineering for at least about 1, 2, 3, 4, 5, 6, 7, 8, 9, 10, 11, 12, 13, 14, 15 or more day(s).

In one embodiment, the animal cell aggregates are cultured on culture inserts for tissue engineering for at least about 1, 2, 3, 4, 5, 6, 7, 8, or more week(s).

For example, in one embodiment, the tissue or tissue-like structures, in particular human tissue or tissue-like structures, are engineered on culture inserts for tissue engineering during an incubation of at least about three weeks at about 37° C. and in about 5% CO₂, wherein during the first 3, 4, 5, 6, 7, 8, 9, or 10 days the medium in both the upper and lower compartments is changed every 2 days, then, optionally, the upper compartment is emptied and the medium in the lower compartment is changed every 2 or 3 days for another 1, 2, 3, or 4 week(s).

The present invention also relates to tissue or tissue-like structure(s) engineered with the Volvox-derived beads of the invention.

According to the present invention, said tissue or tissue-like structures refer to animal tissue or animal tissue-like structures engineered from the free animal cell aggregates comprising or consisting of animal cells and at least one Volvox-derived bead as described hereinabove.

In one embodiment, the tissue or tissue-like structures of the invention thus comprise animal cells and Volvox-derived beads, with some of the animal cells attached or adhering to the Volvox-derived beads. In one embodiment, in the tissue or tissue-like structures of the invention, some animal cells are not in direct contact with the Volvox-derived beads.

In one embodiment, the tissue or tissue-like structures of the invention comprise animal cells and Volvox-derived beads in a ratio of at least about 10, 20, 50, 75, or 100 animal cells for one Volvox-derived bead.

In one embodiment, the tissue or tissue-like structures of the invention comprise animal cells and Volvox-derived beads in a ratio of at least about 100, 250, 500, 750 or 1000 animal cells for one Volvox-derived bead.

In one embodiment, the tissue or tissue-like structures of the invention comprise animal cells and Volvox-derived beads in a ratio of about 1000, 2500, 5000, 7500 or 10000 animal cells for one Volvox-derived bead.

In one embodiment, the tissue or tissue-like structures of the invention comprise animal cells and Volvox-derived beads in a ratio of about 10 to about 10000 animal cells for one Volvox-derived bead.

In one embodiment, the tissue or tissue-like structures of the invention are obtained, or susceptible to be obtained, by the method of engineering tissue or a tissue-like structure as described hereinabove.

Another object of the invention is a composition comprising tissue or a tissue-like structure engineered with the Volvox-derived beads of the invention. Thus, one object of the invention is a composition comprising tissue or tissue-like structure(s) engineered with the Volvox-derived beads of the invention.

Another object of the invention is a pharmaceutical composition comprising tissue or a tissue-like structure engineered with the Volvox-derived beads of the invention and at least one pharmaceutically acceptable excipient. Thus, one object of the invention is a pharmaceutical composition comprising tissue or tissue-like structure(s) engineered with the Volvox-derived beads of the invention and at least one pharmaceutically acceptable excipient.

Another object of the invention is a medicament comprising tissue or a tissue-like structure engineered with the Volvox-derived beads of the invention. Thus, one object of the invention is a medicament comprising tissue or tissue-like structure(s) engineered with the Volvox-derived beads of the invention.

In one embodiment, the composition, pharmaceutical composition, and/or medicament of the invention comprises at least one additional component that will affect the adherence, growth, vascularization, and/or engraftment of the tissue or tissue-like structures engineered with the Volvox-derived beads of the invention.

Examples of said at least one additional component include, without being limited to, silk fibroin, extracellular matrix (ECM) proteins (such as collagen, laminin and/or fibronectin), lymphokines, monokines, growth factors, hormones, corticosteroids, cyclosporin, methotrexate and any mixtures thereof.

In one embodiment, the composition, pharmaceutical composition, and/or medicament of the invention comprises at least one additional component that will provide improved usability or handling characteristics, while not significantly altering the stability or form of the composition, pharmaceutical composition, and/or medicament of the invention.

Examples of said at least one additional component include, without being limited to, antiseptics, antibiotics, antivirals, antioxidants, anti-inflammatory compounds, anaesthetics, plasticizers, preservatives, cell growth medium and/or cryoprotectants.

In one embodiment, the at least one additional component is selected from the group comprising or consisting of metal salts such as a copper salt or a zinc salt, sugar derivatives such as sucralfate, and non-metabolizable sugars.

Pharmaceutically acceptable excipients that may be used in the pharmaceutical composition of the invention include non-toxic solid, semi-solid or liquid filler, diluent, encapsulating material or formulation auxiliary of any type.

The formulation of the composition or pharmaceutical composition or medicament that comprises the tissue or tissue-like structures engineered with the Volvox-derived beads of the invention may be in any form that is suitable for administration, preferably suitable for injection, such as for example, solutions or gels, and said composition or pharmaceutical composition or medicament may be administered by any suitable means, for example, subcutaneous injection, so they will include the carrier(s) or pharmaceutically acceptable excipient(s) necessary to make up the desired form of administration, preferably a form suitable for injection.

The present invention also relates to the cosmetic use of the tissue or tissue-like structure(s) engineered with the Volvox-derived beads of the invention or of the composition or pharmaceutical composition comprising said tissue or tissue-like structure(s) as described hereinabove.

Example of cosmetic uses include, without being limited to, tissue filling, in particular soft tissue filling, such as, for example, lip augmentation or smoothing out of age-related folds, lines, oral commissures or wrinkles.

Thus, in one embodiment, the present invention relates to the use of the tissue or tissue-like structure(s) engineered with the Volvox-derived beads of the invention or of the composition or pharmaceutical composition comprising said tissue or tissue-like structure(s) for soft tissue filling. In other words, in one embodiment, the present invention relates to the use of the tissue or tissue-like structure(s) engineered with the Volvox-derived beads of the invention or of the composition or pharmaceutical composition comprising said tissue or tissue-like structure(s) as soft tissue filler.

Example of soft tissue that may be filled with the tissue or tissue-like structure(s) engineered with the Volvox-derived beads of the invention or with the composition or pharmaceutical composition comprising said tissue or tissue-like structure(s) include, without being limited to, fascia, skin, mucosa, fibrous tissues, and fat.

In one embodiment, the invention relates to the use of the tissue or tissue-like structure(s) engineered with the Volvox-derived beads of the invention or of the composition or pharmaceutical composition comprising said tissue or tissue-like structure(s) for lip augmentation. In another embodiment, the invention relates to the use of the tissue or tissue-like structure(s) engineered with the Volvox-derived beads of the invention or of the composition or pharmaceutical composition comprising said tissue or tissue-like structure(s) for smoothing out age-related folds, lines, oral commissures or wrinkles.

In one embodiment, the tissue or tissue-like structure(s) engineered with the Volvox-derived beads of the invention are obtained from cells of the subject on whom/which the tissue or tissue-like structure(s) are to be used. Thus, in one embodiment, the tissue or tissue-like structure(s) engineered with the Volvox-derived beads of the invention are obtained from autologous animal cells, preferably autologous human cells.

In another embodiment, the tissue or tissue-like structure(s) engineered with the Volvox-derived beads of the invention are obtained from cells of a subject other than the subject on whom/which the tissue or tissue-like structure(s) are to be used. Thus, in another embodiment, the tissue or tissue-like structure(s) of the invention are obtained from allogenic animal cells, preferably allogenic human cells.

The present invention also relates to a cosmetic method for soft tissue filling, said method comprising injecting to a subject the composition or pharmaceutical composition comprising tissue or tissue-like structure(s) engineered with the Volvox-derived beads of the invention as described hereinabove into an area of the body of the subject, thereby filling the tissue at and around said area.

In one embodiment, the composition or pharmaceutical composition to be injected comprises tissue or tissue-like structure(s) engineered with the Volvox-derived beads of the invention obtained from cells of the subject to whom/which the tissue or tissue-like structure(s) are to be injected. Thus, in one embodiment, the composition or pharmaceutical composition to be injected comprises tissue or tissue-like structure(s) engineered with the Volvox-derived beads of the invention obtained from autologous animal cells, preferably autologous human cells.

In another embodiment, the composition or pharmaceutical composition to be injected comprises tissue or tissue-like structure(s) engineered with the Volvox-derived beads of the invention obtained from cells of a subject other than the subject to whom/which the tissue or tissue-like structure(s) are to be injected. Thus, in another embodiment, the composition or pharmaceutical composition to be injected comprises tissue or tissue-like structure(s) engineered with the Volvox-derived beads of the invention obtained from allogenic animal cells, preferably allogenic human cells.

In one embodiment, the composition or pharmaceutical composition comprising the tissue or tissue-like structure(s) of the invention is injected subcutaneously.

In one embodiment, the injection of the composition or pharmaceutical composition comprising the tissue or tissue-like structure(s) of the invention is repeated over time. In one embodiment, the injection of the composition or pharmaceutical composition comprising the tissue or tissue-like structure(s) of the invention is repeated one, two, three, four, five, six, seven, eight, nine or ten times. In one embodiment, the injection of the composition or pharmaceutical composition comprising the tissue or tissue-like structure(s) of the invention is repeated one week, two weeks, three weeks, four weeks, or one month after the first injection.

In one embodiment, the area of the body of the subject into which the composition or pharmaceutical composition comprising the tissue or tissue-like structure(s) of the invention is injected is comprised within the facial and neck area. In one embodiment, the composition or pharmaceutical composition comprising the tissue or tissue-like structure(s) of the invention is injected in the lips.

The present invention also relates to the use of the tissue or tissue-like structure(s) or of the composition, or pharmaceutical composition of the invention as a medicament. Thus, another object of the invention is the tissue or tissue-like structure(s) of the invention, composition, or pharmaceutical composition comprising said tissue or tissue-like structure(s) for use as a medicament.

According to the present invention, the tissue or tissue-like structure(s) of the invention, composition, pharmaceutical composition or medicament comprising said tissue or tissue-like structure(s) is/are to be administered, preferably to be injected, to a subject in need thereof.

In one embodiment, the tissue or tissue-like structure(s) engineered with the Volvox-derived beads of the invention are obtained from cells of the subject to whom/which the tissue or tissue-like structure(s) are to be injected and the composition, pharmaceutical composition or medicament of the invention to be injected comprises tissue or tissue-like structure(s) engineered with the Volvox-derived beads of the invention obtained from cells of the subject to whom/which the composition is to be injected. Thus, in one embodiment, the tissue or tissue-like structure(s) engineered with the Volvox-derived beads of the invention are obtained from autologous animal cells, preferably autologous human cells and the composition, pharmaceutical composition or medicament of the invention to be injected comprises tissue or tissue-like structure(s) engineered with the Volvox-derived beads of the invention obtained from autologous animal cells, preferably autologous human cells.

In another embodiment, the tissue or tissue-like structure(s) engineered with the Volvox-derived beads of the invention are obtained from cells of a subject other than subject to whom/which the tissue or tissue-like structure(s) are to be injected and the composition, pharmaceutical composition or medicament of the invention to be injected comprises tissue or tissue-like structure(s) engineered with the Volvox-derived beads of the invention obtained from cells of a subject other than the subject to whom/which the composition is to be injected. Thus, in another embodiment, the tissue or tissue-like structure(s) engineered with the Volvox-derived beads of the invention are obtained from allogenic animal cells, preferably allogenic human cells and the composition, pharmaceutical composition or medicament of the invention to be injected comprises tissue or tissue-like structure(s) engineered with the Volvox-derived beads of the invention obtained from allogenic animal cells, preferably human allogenic cells.

Another object of the invention is the tissue or tissue-like structure(s) of the invention, composition, pharmaceutical composition, or medicament comprising said tissue or tissue-like structure(s) for use in the treatment of tissue loss or injury.

According to the present invention, the tissue loss or injury may be the loss or injury of soft tissue, bone or cartilage.

Examples of soft tissues include, without being limited to, tendons, ligaments, fascia, skin, mucosa, fibrous tissues, fat, synovial membranes, muscles, nerves and blood vessels.

According to one embodiment, tissue loss or injury is inborn or is the consequence of a disease, a treatment, a surgery or a trauma.

In one embodiment, the tissue or tissue-like structure(s) of the invention, composition, pharmaceutical composition, or medicament comprising said tissue or tissue-like structure(s) are for use in the treatment of an inborn tissue loss or injury, for example a tissue loss or injury resulting from the failure of a tissue to develop normally.

In one embodiment, the tissue or tissue-like structure(s) of the invention, composition, pharmaceutical composition, or medicament comprising said tissue or tissue-like structure(s) are for use in the treatment of tissue loss or injury resulting from a disease.

Examples of diseases that may result in tissue loss or injury include, without being limited to, scleroderma, diabetes, vasculitis, muscle diseases (such as, for example, inflammatory muscle diseases, neurogenic muscle diseases, myogenic muscle diseases, muscular dystrophies, congenital myopathies, or myasthenia gravis), vascular diseases (such as, for example, peripheral arterial diseases, abdominal aortic aneurysms, carotid diseases, or venous diseases).

In one embodiment, the tissue or tissue-like structure(s) of the invention, composition, pharmaceutical composition, or medicament comprising said tissue or tissue-like structure(s) are for use in the treatment of tissue loss or injury resulting from a treatment.

Example of treatments that may result in tissue loss or injury include, without being limited to, radiation treatment (also referred to as radiotherapy), such as cancer radiation treatment.

In one embodiment, the tissue or tissue-like structure(s) of the invention, composition, pharmaceutical composition, or medicament comprising said tissue or tissue-like structure(s) are for use in the treatment of tissue loss or injury resulting from surgery or trauma. Thus, in other words, in one embodiment the tissue or tissue-like structure(s) of the invention, composition, pharmaceutical composition, or medicament comprising said tissue or tissue-like structure(s) are for use in the treatment of post-surgical and/or post-traumatic tissue loss or injury.

Examples of post-traumatic tissue losses or injuries include, without being limited to, tissue losses or injuries resulting from a wound, a burn, an ulcer (such as a venous leg ulcer, a skin ulcer, a diabetic ulcer).

The present invention also relates to a method for treating tissue loss or injury as described hereinabove in a subject in need thereof, said method comprising injecting to the subject the tissue or tissue-like structure(s) of the invention, the composition, the pharmaceutical composition or the medicament comprising said tissue or tissue-like structure(s).

In on embodiment, said injection is a subcutaneous injection.

In one embodiment, the method of treatment of the invention is for treating post-surgical and/or post-traumatic tissue loss or injury.

In one embodiment, the method of treatment of the invention comprises:
culturing cells previously obtained from the subject with the method of the invention as described hereinabove, thereby obtaining free animal cell aggregates comprising or consisting of animal cells and at least one Volvox-derived bead;
transferring said free animal cell aggregates on culture inserts for tissue engineering and culturing said animal cell aggregates, thereby obtaining tissue or tissue-like structures;
administrating the tissue or tissue-like structures to the subject.

It will be understood that the total daily usage of the tissue or tissue-like structure(s) of the invention, composition, pharmaceutical composition, or medicament comprising said tissue or tissue-like structure(s) will be decided by the attending physician within the scope of sound medical judgment. The specific therapeutically effective amount for any particular subject will depend upon a variety of factors including the tissue loss or injury being treated and the severity of the tissue loss or injury; activity of the specific agent employed; the specific composition employed, the age, body weight, general health, sex and diet of the patient; the duration of the treatment; drugs used in combination or coincidental with the specific agent employed; and like factors well known in the medical arts. For example, it is well within the skill of the art to start with an amount lower than that required to achieve the desired therapeutic effect and to gradually increase the amount until the desired effect is achieved.

The present invention also relates to artificial organs engineered with the Volvox-derived beads of the invention.

According to the present invention, said artificial organs are engineered from free animal cell aggregates comprising or consisting of animal cells and at least one Volvox-derived bead as described hereinabove.

Without wishing to be bound to any theory, the Applicants suggest that using Volvox-derived beads of different sizes, for example through the use of different Volvox species, with the different types of cells that are found within an organ, may facilitate the three-dimensional organization of the different types of cells within a volume and thereby allow the engineering of an artificial organ with an architecture mimicking that of the corresponding native organ.

The Volvox-derived beads of the invention and their use, in particular in the method of culturing animal cells of the invention, enable animal cells to grow and proliferate after adhesion to said beads, thus resulting in the formation of free aggregates comprising or consisting of animal cells and Volvox-derived beads. Said aggregates form three-dimensional structures which can be further cultured to obtain tissue or a tissue-like structure, in particular with the method of engineering tissue or a tissue-like structure of the invention.

The Volvox-derived beads of the invention thus lead to the engineering of tissue or tissue-like structure(s) without the requirement for animal-based substrate, such as the commonly used type I collagen.

As demonstrated in the Examples, the tissue-like structures of the invention are suitable for in vivo implantation and do not cause any untoward reaction such as necrosis or inflammation of the surrounding tissue. Inflammation reactions are often associated with unwanted outcomes of dermal insertions, such as cysts or nodules development (Ledon et al., Am J Clin Dermatol. 2013 October; 14(5):401-11).

Implantation of the tissue-like structures of the invention results in a stable filling of the surrounding tissue, with a progressive colonization of the interstitial spaces by stromal ingrowths associated with the beginning of a vascularization of the area observable one month after implantation. The visible presence of blood vessels in the area augmented following subcutaneous implantation is the mark of the successful integration of the implanted structures inside the recipient body (Beleznay et al., J Clin Aesthet Dermatol. 2014 September; 7(9):37-43).

Moreover, no rapid degradation or collapse of the tissue-like structures of the invention was observed following in vivo implantation. By contrast, collagen gels of bovine origin used as a control were partially degraded only three weeks after implantation and completely degraded three months after implantation.

The Volvox-derived beads of the invention may also have other uses, such as, for example, in the cryopreservation of animal cells and in the manufacture of ascites for antibody production.

Additionally, the Applicants observed that the Volvox-derived beads of the invention may be dehydrated. Rehydration of the dehydrated Volvox-derived beads does not induce any change in their morphological and structural properties, as compared to Volvox-derived beads that did not undergo the dehydration/rehydration process.

Dehydrated Volvox-derived beads offer additional advantages, notably increased conservation, easier conservation and easier transport.

### BRIEF DESCRIPTION OF THE DRAWINGS

FIG. 1 is a group of photographs obtained with a Leica DMI1 inverted microscope equipped with a Leica MC170HD camera showing Volvox colonies. FIG. 1A is a photograph of living Volvox colonies and FIG. 1B is a photograph of Volvox colonies inactivated following a 24 h incubation in ethanol, bar: 100 μm.

FIG. 2 is a graph showing the viability assessed with a MTS cytotoxicity assay of L929 mouse fibroblasts following incubation with medium (negative control), a pure Volvox extract (100%), or toxic extracts (positive control) either pure (100%) or diluted (50%). Cell viability was expressed as a percentage of the number of viable cells after incubation in the extract considered with respect to the number of viable cells after incubation in medium (negative control).

FIG. 3 is a group of photographs showing VCBs before (FIG. 3A) and after (FIGS. 3B, C) seeding with L929 mouse fibroblasts. The photographs of FIGS. 3A and B were obtained with a Leica DMI1 inverted microscope equipped with a Leica MC170HD camera, bar 100 μm. White arrows show the L929 mouse fibroblasts attached at the surface of a VCB after 2 h of incubation (FIG. 3B). The photograph of FIG. 3C was obtained with an environmental scanning electron microscope (ESEM) and show the aggregation of mouse fibroblasts at the surface of a VCB, bar 10 μm.

FIG. 4 is a is a group of photographs showing human dermal fibroblasts seeded either on alginate beads (FIG. 4A) or on VCBs consisting of inactivated Volvox colonies (FIG. 4B). The floating aggregates of human dermal fibroblasts and VCBs were transferred on culture inserts for further incubation (FIG. 4C), leading to the engineering of a tissue-like structure (FIG. 4D). A cross-section of the tissue-like structure with a thickness up to 300 μm was observed after detection of Volvox-beads through a chemical coupling with rhodamine and of fibroblast nuclei labelled with DAPI (FIG. 4E) and after detection with hematoxylin eosin saffron staining (FIG. 4F). Bars as indicated. Photograph were obtained with a Leica DMI1 inverted microscope equipped with a Leica MC170HD camera (FIGS. 4A and B), a canon IXUS 175 camera (FIGS. 4C and D), a Leica DMI600 fluorescence microscope equipped with a DFC300FX camera (FIG. 4E) and a Leica DM1000 optical microscope equipped with a DMC 4500 camera (FIG. 4F).

FIG. 5 is a is a group of photographs showing the tissue augmentation immediately (FIG. 5A) or one month (FIG. 5B) after subcutaneous injection to a nude mouse of VCB-engineered tissue-like structures. The photographs of FIGS. 5A and B were obtained with a Canon IXUS 175 camera, black arrows indicate the tissue augmentation resulting from the injection. Tissue analysis was carried out on a biopsy performed one month after injection of bovine collagen gel (FIG. 5C) or VCB-engineered tissue-like structures (FIG. 5D). The photographs of FIGS. 5C and D were obtained with a Leica DMS1000 macroscope, bar 500 μm. Histological analysis with hematoxylin eosin saffron staining was carried out on the biopsy specimen (FIGS. 5E, F, G, H). The photographs of FIGS. 5E, F, G and H were obtained with an inverted optical microscope, bar as indicated. Black arrow indicates the presence of blood vessels (FIG. 5H).

### EXAMPLES

The present invention is further illustrated by the following examples.

### Example 1

Materials and Methods

Material

Volvox carteri strain (NIES-397) was obtained from the Microbial Culture Collection of the National Institute for Environmental Studies in Japan. The algae were grown in VT medium at 22° C. with a day/night cycle of 12 h/12 h and lighting of 13000 lux and 37 W/m². VT medium was prepared according to Provasoli et al., 1959 and comprises 500 μmol/L Ca(NO₃)₂; 235 μmol/L Na₂-βglycerophosphate; 162 μmol/L MgSO₄; 670 μmol/L KCl; 0.07 nmol/L vitamin B12; 0.41 nmol/L biotin; 30 nmol/L thiamine; 3.8 mmol/L glycylglycine; 8 μmol/L Na₂EDTA.2H₂O; 2.2 μmon FeCl₃; 0.55 μmon MnCl2; 0.11 μmon ZnSO₄; 0.05 μmon CoCl₂; 0.036 μmol/L Na₂MoO₄; pH is buffered at 7.5.

L929 mice fibroblasts (ATCC, reference ATCC® CCL-1™) and neonatal human dermal fibroblasts (HDFn, Thermo Fisher Scientific, catalog number C-004-5C) were grown in DMEM medium supplemented with 10% FCS (Hyclone), 2 mM L-glutamine, 100 U/mL penicillin and 100 μg/mL streptomycin, thereafter referred to as complete culture medium, at 37° C. under humidified atmosphere (5% CO₂, 95% air).

Alginate beads were produced from an alginate solution (MANUCOL® LKX FMC Biopolymer 1.5% in 0.9% NaCl solution) by an extrusion method using an air-jet droplet generator system. The beads were gelled in a CaCl₂ bath (120 mM in 0.9% NaCl solution, pH 7.6). Cross-linked beads were washed with PBS and stored at 4° C.

Unless otherwise specified, all products were obtained from Gibco.

Methods

Preparation of Volvox-Derived Carrier Beads (VCBs)

The Volvox colonies were sieved on a stainless-steel screen with a porosity of 100 μm, then washed with PBS and centrifuged at 300 g for 5 minutes at room temperature. Volvox-derived beads, hereafter referred to as Volvox-derived carrier beads (VCBs), were obtained by incubation of Volvox colonies in 70% ethanol for at least 2 hours at 4° C.

Culture of L929 Cells with VCBs

A pellet of about 1 cm³ of VCBs was mixed with 1.10⁶ L929 cells in 1 mL of complete culture medium in a sterile tube. The mixture was incubated at 37° C. for 45 minutes before being transferred in a polystyrene Petri dish untreated for cell culture and further incubated for 24 h at 37° C. under humidified atmosphere (5% CO₂, 95% air). Images of the culture were taken using an inverted optical microscope. The cells were rinsed in PBS, fixed in 3% glutaraldehyde in Rembaum buffer (pH 7.4) for 1 h and observed with an environmental scanning electron microscope (ESEM).

Culture of HDFn Cells with VCBs

A pellet of about 1 cm3 of VCBs was mixed with 1.10⁶ HDFn cells in 1 mL of complete culture medium in a 15-mL centrifuge tube. The mixture was incubated at 37° C. for 45 minutes before being transferred to a polystyrene Petri dish untreated for cell culture and further incubated for 24 h. The cells were then transferred into a 23-mm diameter polycarbonate culture insert (Nunc). 2 mL of complete culture medium were added to the upper compartment and 1 mL was added in the lower compartment. The cells were grown at 37° C. under humidified atmosphere (5% CO₂, 95% air). The media was changed every 2 days for 6 days, then the upper compartment was emptied. Cells were fed with 1 mL of culture medium in the lower compartment. This medium was renewed every 2 days for two additional weeks.

Implantation of Tissue-Like Structures in Athymic Mice

All grafting experiments were done according to an animal protocol approved by the local ethic comity for animal use. A tissue-like structure engineered from VBCs seeded with human dermal fibroblasts was collected and injected subcutaneously in the back of an athymic mouse through a sterile syringe. The animal was sacrificed after one month and the skin at implantation point was harvested and observed under a macroscope. The biopsy was afterwards fixed for 1 h in 4% formalin buffer, dehydrated in a series of graded alcohols, and included in paraffin. Blocks were sectioned at 8 μm thickness using a microtome and slides were stained using a standard hematoxylin and eosin staining protocol.

Cytokine Secretion

Cytokine secretion was measured from culture supernatants at different times following the seeding of VCBs with cells. Human interleukin 6 (IL6) ELISA ready-set-go (eBiosciences) was used according to the manufacturer's instructions. Standard curves were plotted simultaneously from serial dilutions of recombinant human IL6 included in the kit.

Toxicity Test

A pure Volvox extract (100%) was obtained by incubating a pellet of about 1 cm3 of fixed Volvox colonies in 1 mL of DMEM at 37° C. under agitation for 24 h. Both a negative control, i.e., a non-toxic extract corresponding to medium only, and a positive control, i.e., a toxic extract, were obtained in parallel. DMEM (6 mL/cm²) was added both to Thermanox coverslips (non-toxic extract) and to the wells of a plate which bottom was covered with a self-curing luting composite (toxic extract).

The extracts were then deposited on monolayers of L929 fibroblasts seeded the day before in the wells of 96-well plates. The culture was extended by 24 h at 37° C. in the presence of 5% CO₂.

A MTS cytotoxicity assay was carried out to assess the toxicity of the extracts. The assay is based on the reduction of MTS tetrazolium compound by viable cells to generate a colored formazan product that is soluble in cell culture media. This conversion is thought to be carried out by NAD(P)H-dependent dehydrogenase enzymes in metabolically active cells. The formazan dye produced by viable cells can be quantified by measuring the absorbance at 490-500 nm. A decrease in the absorbance at 490-500 nm reflects a decrease in the metabolic activity correlated to a decrease in the number of living cells. Following incubation with the extracts, 20 μL, of MTS reagent were thus dispensed in each well and incubated for 2 h at 37° C. The absorbance at 490 nm was measured and the cell viability was determined. Cell viability was expressed as a percentage of the number of viable cells after incubation in the extract considered with respect to the number of viable cells after incubation in the non-toxic extract (negative control). In accordance with the ISO10993 standards, an extract associated with a cell viability higher than 70% was considered as non-toxic.

Results

Volvox-Derived Carrier Beads

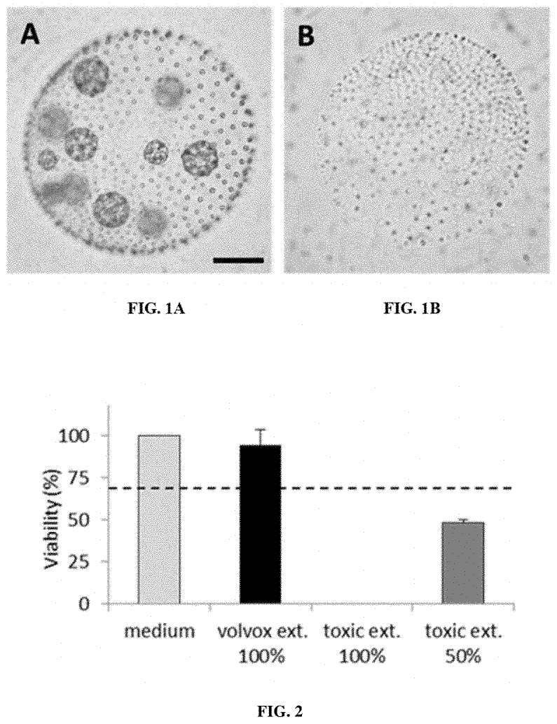

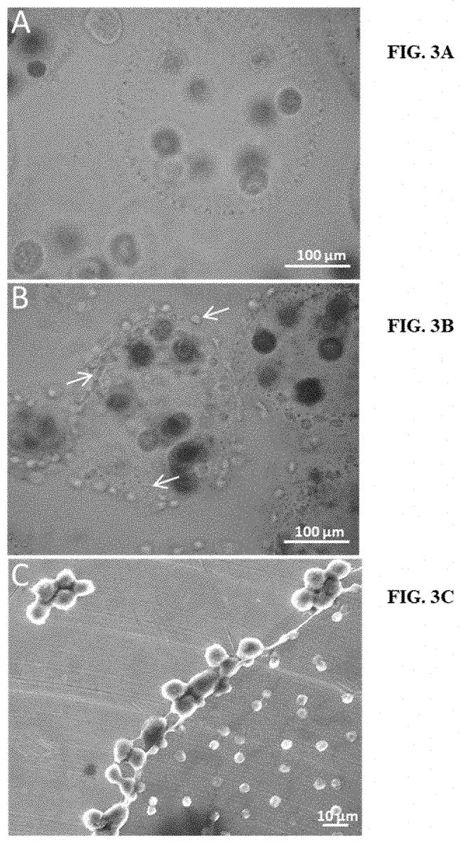

Volvox algae were cultivated using standard techniques and their viability was monitored through their capacity to roll and proliferate in the culture conditions (see FIG. 1A). Volvox colonies were collected by filtration and the medium was replaced with 70% ethanol, thus killing all the algae cells of the Volvox colonies (see FIG. 1B and FIG. 3A). After 12 hours in ethanol, the Volvox colonies lost their ability to float in the culture medium. No rolling could be detected and dead colonies settled at the bottom of the culture dish under the action of gravity. Further attempts to revivify the Volvox colonies through an incubation in fresh culture medium were unsuccessful, demonstrating that no living cells were present after ethanol treatment. The obtained entities were named Volvox-derived beads or Volvox-derived carrier beads (VCBs). During the incubation in ethanol, the Volvox colonies lost most of their chlorophyll pigments, which diffused into the ethanol. Some pigments were still present in the daughter colonies embedded in the mucus core of the VCBs. These pigments were found to be non-toxic for all the cell types tested. Moreover, these internal pigmented dots proved to be useful as a rapid detection means of the mucus core of VCBs when mammalian cells were cultured in the presence of VCBs.

Volvox-Derived Carrier Beads as a Support for Mammalian Fibroblast Culture

In order to test the capacity of the VCBs to serve as a cell-colonizable substrate, L929 fibroblasts, i.e., a murine cell line originating from subcutaneous tissue, were cultured at their contact (see FIG. 3). These cells are the cells of choice for cytotoxicity standardized tests such as the ISO 10993 tests. The L929 fibroblasts rapidly adhered to the globular structures of the VCBs. Indeed, after 2 hours, some fibroblasts could be seen to have attached to the outside part of the VCBs (FIG. 3B). The colonization of the VCBs continued until they were completely covered with fibroblasts. The morphology of the attached fibroblasts was examined using an environmental scanning electron microscope (ESEM). They formed small aggregates (FIG. 3C) of two or three cells tightly attached to the surface of the VCBs without any respect to the former cellular niche abridging the original algal cells. No fibroblasts could be seen penetrating the inner compartment of the VCBs.

In conclusion, the VCBs were able to efficiently promote cell attachment and proliferation at their surface, thus leading to the formation of cell aggregates.

The suitability of the VCBs as an animal cell culture support was assessed by measuring the viability percentage of the fibroblasts after their contact with Volvox extracts in a MTS cytotoxicity assay (FIG. 2). The viability of the fibroblasts after incubation with a pure Volvox extract was compared to that of the fibroblasts after incubation with a control toxic extract either pure (100%) or diluted (50%). The results of the MTS tests showed a viability of about 95% (94.2%±9.3) after incubation with the pure Volvox extract, while incubation with a pure toxic extract (100%) resulted in a complete loss of viability and incubation with a diluted toxic extract (50%) resulted in a viability of about 50% (48.3%±1.7). A viability of about 95% is markedly greater than the accepted threshold of 70% as defined according to the ISO 10993 standards, thus establishing that the pure Volvox extract is not toxic to the cells.

In conclusion, the inactivated Volvox colonies, i.e., the Volvox-derived carrier beads, make a suitable support for animal cell culture with no observed adverse effects on the viability of said animal cells.

Volvox-Derived Carrier Beads as a Support for the Production of Tissue-Like Structures

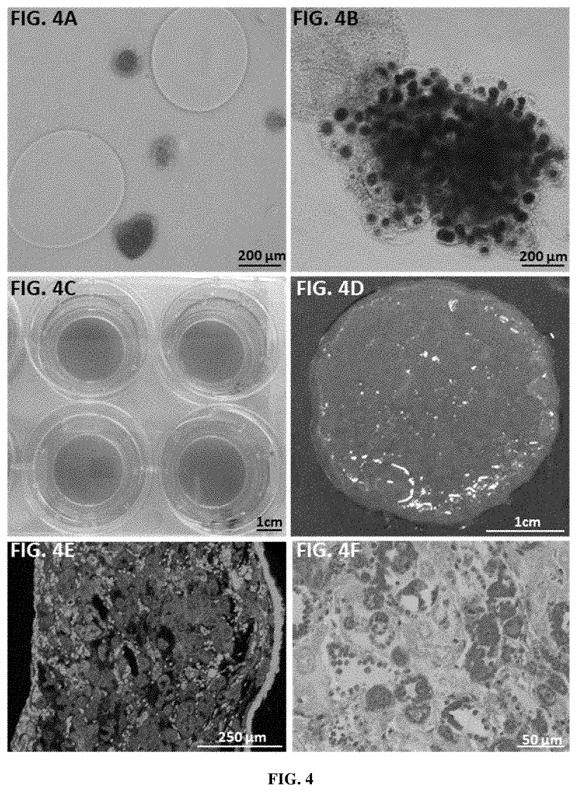

The capacity of VCBs to support the growth of human dermal cells was then tested (see FIG. 4). For this purpose, neonatal dermal fibroblasts from human foreskin (HDFn) were seeded both on alginate beads made of alginic acid, a polymer purified from brown algae, and on VCBs obtained following inactivation of Volvox colonies as described hereinabove.

A comparison between the number of human fibroblasts observed at the surface of alginate beads and that observed at the surface of VCBs demonstrated the outstanding ability of VCBs to enable cell adhesion and growth at their surface (see FIGS. 4A and B). Indeed, while HDFn cells adhered to each other without interacting in any way with the alginate beads (FIG. 4A), HDFn cells adhered rapidly to the VCBs and aggregates of groups of HDFn cells and VCBs could be observed (FIG. 4B).

In conclusion, the inactivated Volvox colonies, i.e., the Volvox-derived carrier beads, proved to be a better support for the growth and proliferation of animal cells than the commonly used alginate beads.

The floating aggregates made of groups of HDFn cells and VCBs were then transferred and cultured into culture inserts used for tissue engineering (FIG. 4C), allowing to continue this aggregation for at least three weeks. A cohesive, tissue-like structure, presenting a jelly texture, was thus obtained (FIG. 4D).

After 21 days of growth in vitro, the cultures presented a tissue-like organization (FIG. 4E) with a width varying between 50 to 300 microns depending on the quantity of HDFn cells-VCBs added in the culture wells. The VCBs could be detected through a chemical coupling with rhodamine Staining with hematoxylin and eosin demonstrated the presence of HDFn cells throughout the whole structure (FIG. 4F). No shrinkage of the tissue-like structures could be observed, as is usually observed with classical collagen-based tissues. The presence of neo-synthesized collagen was assessed by a Hematoxylin-eosin-saffron trichromatic staining in these blocks. Inside the three-dimensional structures, the fibroblasts appeared flat growing between large blocks of VCBs (FIGS. 4E and 4F). No apoptotic cells could be detected in these tissues. Finally, a measurement of the pro-inflammatory interleukin 6 (IL-6) released in the culture medium was done every two to three days for a total of two weeks of culture. During this time, no IL-6 secretion could be detected, reinforcing the non-toxicity of the VCBs and demonstrating their role as a passive but effective support to grow and carry animal cells.

In conclusion, the inactivated Volvox colonies, i.e., the Volvox-derived carrier beads, allows the engineering of healthy tissue-like structures which do not show any sign of inflammation or apoptosis.

In Vivo Transplantation of Tissue-Like Structures Engineered with Volvox-Derived Carrier Beads

In order to test their in vivo behavior, three-dimensional tissue-like structures of human dermal fibroblasts obtained as described hereinabove were subcutaneously implanted in athymic mice.

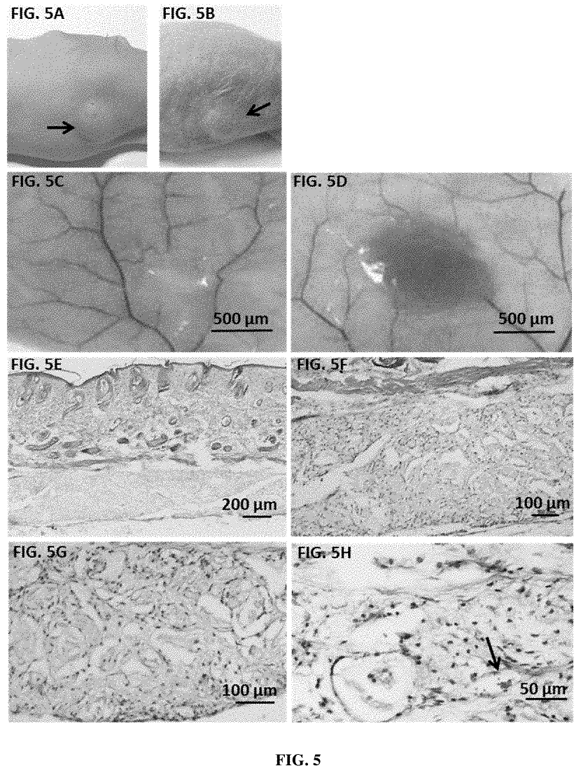

To this aim, the tissues were collected in a syringe and a total volume of 150 mm³ of jelly-like substance was injected directly under the mouse skin creating a protruding implant visible from the outside (FIG. 5A). The volume augmentation created by the injection remained detectable and unchanged in size and shape during the totality of the experiment (FIG. 5B). As a control, an equal amount of bovine collagen gel was similarly injected to mice in order to create a similar soft tissue augmentation.

After one month, at necropsy, histological analysis of biopsy specimens taken from mice implanted with the VCBs-engineered tissues showed the presence of a large mesenchymal tissue-like structure penetrated by surrounding blood vessels (FIG. 5D). By contrast, histological analysis of biopsy specimens taken from mice injected with an equal amount of bovine collagen gel showed a smaller mass that was hardly detectable under the mouse skin (FIG. 5C). Histological analyses of the biopsy specimens taken from mice implanted with the VCBs-engineered tissues also demonstrated that the implanted tissue-like structures and surrounding tissues showed no signs of inflammation, as shown by the absence of eosinophils, neutrophils and macrophages or any other inflammatory cells (FIGS. 5E, F and G). Moreover, the histological analyses showed a progressive colonization of the interstitial spaces by stromal ingrowths associated with the beginning of a vascularization of the area one month after grafting (FIG. 5H arrow).

In conclusion, the inactivated Volvox colonies, i.e., the Volvox-derived carrier beads, allows the engineering of tissue-like structures which are suitable for in vivo implantation. Indeed, implantation of said VCB engineered tissue-like structures does not induce any inflammation of the surrounding tissues. Moreover, after implantation, the VCB engineered tissue-like structures remain stable, with no sign of rapid degradation or collapse, and actually show sign of being well-integrated, as indicated by the observation of the beginning of a vascularization.

1-15. (canceled)

16. A composition, or solid support, or scaffold for animal cell culture, comprising Volvox-derived beads, said Volvox-derived beads consisting of inactivated Volvox colonies.

17. A method of culturing animal cells comprising culturing the animal cells in a culture medium comprising a composition, or solid support, or scaffold for animal cell culture, comprising Volvox-derived beads according to claim 16, wherein the animal cells adhere to the Volvox-derived beads and proliferate around said Volvox-derived beads, thereby forming animal cell aggregates comprising animal cells and at least one Volvox-derived bead.

18. The method according to claim 17, wherein said method comprises:
first contacting the animal cells to be cultured with the composition, or solid support, or scaffold for animal cell culture, comprising Volvox-derived beads, and incubating them in culture medium;
then transferring the animal cells and the composition, or solid support, or scaffold for animal cell culture, comprising Volvox-derived beads, to a culture vessel and culturing the animal cells in the presence of the composition, or solid support, or scaffold for animal cell culture, comprising Volvox-derived beads.

19. The method according to claim 17, wherein the animal cells to be cultured and the Volvox-derived beads are contacted in a ratio of about 1.10⁶ animal cells for about 0.2 cm³ to about 4 cm³ Volvox-derived beads.

20. The method according to claim 17, wherein the animal cells to be cultured are human cells.

21. The method according to claim 17, wherein the animal cells to be cultured are human primary cells.

22. A free animal cell aggregate comprising animal cells and at least one Volvox-derived bead, said Volvox-derived bead consisting of an inactivated Volvox colony.

23. A method of engineering tissue or a tissue-like structure, comprising:
culturing animal cells in a culture medium comprising a composition, or solid support, or a scaffold for animal cell culture, comprising Volvox-derived beads according to the method of claim 17, thereby obtaining free animal cell aggregates comprising animal cells and at least one Volvox-derived bead, said Volvox-derived bead consisting of an inactivated Volvox colony;
transferring and culturing said animal cell aggregates on culture inserts for tissue or tissue-like engineering and culturing said animal cell aggregates, thereby obtaining tissue or a tissue-like structure.

24. A composition comprising tissue or a tissue-like structure engineered from the free animal cell aggregate of claim 22.

25. A pharmaceutical composition comprising tissue or a tissue-like structure engineered from the free animal cell aggregate of claim 22, and at least one pharmaceutically acceptable excipient.

26. A method of filling tissue in a subject in need thereof, comprising injecting to the subject the composition according to claim 24.

27. The method according to claim 26, wherein said method is for a cosmetic treatment.

28. The method according to claim 26, wherein said method is for the treatment of tissue loss or injury.

29. The method according to claim 28, wherein the tissue loss or injury is a post-surgical and/or a post-traumatic tissue loss or injury.

30. A method of producing Volvox-derived beads, comprising:
culturing Volvox algae in order to obtain Volvox colonies;
isolating the Volvox colonies; and
inactivating the Volvox colonies, thereby producing Volvox-derived beads consisting of inactivated Volvox colonies.

31. The method according to claim 30, wherein the Volvox colonies are inactivated in ethanol at a concentration expressed in volume/volume percent (v/v) ranging from about 60% to about 80%, for at least about 1 h, at about 4° C.

32. The method according to claim 31, wherein the Volvox colonies are inactivated in ethanol at a concentration expressed in volume/volume percent (v/v) ranging from about 65% to about 75%.

33. The method according to claim 31, wherein the Volvox colonies are inactivated in ethanol for at least about 2 h.

34. The method according to claim 30, further comprising dehydrating the Volvox-derived beads, thereby obtaining dehydrated Volvox-derived beads.


SEQUENCE LISTING (6 sequences)

SEQ ID NO: 1 - DNA, 310 bp, Volvox carteri f. nagariensis
aaatctcaatgccgtacacacgcatgatgatgcatgtgtacgacattggatcttgttttctcggcattaaatcatttactcaaatcgtttaatgccgggtgaattgaagtatgtagatgcttaaccgtggaccgtttcatcgggcctttactgggtaggcaaagtcagcgttatgtaatgcatacgttgcttgctgcaatgctttagtagatggctttgaggttctgtgtttcgcaaaaaatcaggattactgttgtactgcacgggactctgtagtactgtgtagtacagcaagtgtactatttttttc

SEQ ID NO: 2 - DNA, 307 bp, Volvox carteri f. nagariensis
aaatctcaatgccgtacacacgcatgatgcacgtgtacgacattggatcttgttttctcggcattaaatcatttactcaaatcgtttaatgccgggtgaattgaagtatgtagatgcttaaccgtggaccgtttcatcgggcctttactgggtaggcaaagtcagcgttatgtaatgtatacgttgcttgctgcaatgctttagtagatggctttgaggttctgtgtttcgcaaaaaatcaggattactgttgtactgcacaggactctgtagtactgtgtagtacagcaagtgtactatttttttc

SEQ ID NO: 3 - DNA, 272 bp, Volvox carteri f. kawasakiensis
aaaactcagtgccgtacacgcatatgtgtgtgtgtgtgtacgacattggatctggttttctcggcattaaacttttatataaaatctttaatgtgccgggtggactgaagtgtgtagatgctgaaccatggaccgtattatctcgggcctctagtgggtaggcaaagtcaaccttaagtgcgttgtttgctgcattgctttagtagatggctttgaggtcctgtgcttggcaaaaaaacaggatatattttaatctaaacaacattttttta

SEQ ID NO: 4 - DNA, 455 bp, Volvox carteri f. weismannia
aaatctcagtgccgtacatgcatctgtgtgtacggcattggatctggtttcctcggcacatacaaatcctttgtttatgtttgaattatagtaataataataattcgaacacctaactctgtgagatatgccgggtgaaccgaagtatacagatgctggatcgtggaccgttgatatttatatatcgggcctctactgggtaggcaaaatcaacctgtagcagtggtctatactatactatgctctactactgtgttgtttgctgcaaaatgctttagtagatggctttggggtcctgtgtttggcaaaaaagcaggagtgtacgtggctacaatacgtatctgctctgcacttgagtgtgtgctgtactacaacttaaacgtgtgcagtacacaccatgtgtggtgtgcatgtgttgtcgtcgtacatcattatatatatatataattattttc

SEQ ID NO: 5 - DNA, 342 bp, Volvox carteri f. weismannia
aaagctcatcgccgtacatgcacatgtgtgtacggcgttggacctggttttctcggcacatacagatcttttatatgtttaataataacgtataactgaggggtgccgggtgaactgaagtatgcagatgctggaccgtggaccatatactatcgggcctctactgggtaggcaaaatcaacctttgttactactgtgttgtttgctgtgatgctttagtagatggctttggggtcctgtgtttggcaaaaaagcaggagtatgcgactctcgacacgcgtgtgctgtactttgtgcagtacaaaccatatgtgtatgtgttgttgtactttttatttttcc

SEQ ID NO: 6 - DNA, 240 bp, Volvox globator
aaatctcatccagagggagggacatgtggtggttgtgcgatggaactggctgtcccgcatttttgcttgcacgtcaggcaaataaaagcgggtcagctgaagtacagctgttgctgatggaccctacaacgggcctctactgggtaggcaactcgcttgctacaatgctttagtaggtggctttgggactgtgagcagcacgaatcaggattactcgtcatgagttaaaacccctttttc